(19) United States
(12) Patent Application Publication
(10) Pub. No.: US 2026/0057962 A1
(43) Pub. Date: Feb. 26, 2026

(54) Gene Profiling and Candidate Gene Prioritization Using Large Language Models

(71) Applicant: The Jackson Laboratory, Bar Harbor, ME (US)

(72) Inventors: Damian Chaussabel, Bar Harbor, ME (US); Taushif Khan, Bar Harbor, ME (US)

(21) Appl. No.: 19/306,228

(22) Filed: Aug. 21, 2025

Related U.S. Application Data
(60) Provisional application No. 63/686,263, filed on Aug. 23, 2024.

(51) Int. Cl. G16B 20/00; G06F 30/27; G16B 40/20

(52) U.S. Cl. CPC G16B 20/00; G06F 30/27; G16B 40/20

(57) ABSTRACT

The present disclosure relates to a multi-phase method for determining a set of candidate genes. During a first phase, the method includes prompting a naïve language model with a plurality of prompts corresponding to a plurality of candidate genes to generate a set of initial scores indicative of each corresponding candidate gene's potential as a biomarker or therapeutic target. During a second phase, the method includes determining, for each candidate gene, a set of relevant documents from a curated document library. The method also includes prompting a further language model using the relevant documents to generate secondary scores. During a third phase, the method includes determining, for each candidate gene, at least one of: a decision classification, a recalibrated score, and a detailed scientific explanation. The method includes determining a final candidate set and conducting a multi-dimensional optimization analysis on each candidate gene of the final candidate set.

### CROSS-REFERENCE TO RELATED APPLICATION

The present application claims the benefit of U.S. Patent Application No. 63/686,263, filed Aug. 23, 2024, the content of which is herewith incorporated by reference.

### BACKGROUND

Over the past few decades, large-scale profiling techniques have become pivotal in contemporary biomedical research, marked by the advent of multimodal analyses (multi-omics) and single-cell profiling technologies. These advancements, alongside a significant reduction in sequencing costs, have made it possible to measure a vast array of parameters simultaneously. As a result, these techniques have become increasingly accessible, leading to their widespread use across various fields of biomedical research. Such analyses typically yield complex signatures, encompassing tens to thousands of analytes, presenting a significant interpretative challenge. This challenge is further compounded by the need to assimilate the extensive, often unstructured literature associated with these gene or analyte collections.

To address the complexities of interpreting large-scale profiling data, standardized annotations or definitions for each analyte are indispensable. However, traditional tools such as ontology or pathway enrichments, while useful, often provide only a surface-level understanding of the data. These methods tend to be generic and fail to incorporate the nuanced biological or experimental contexts that are critical for a deeper understanding of gene functions and interactions. Consequently, functional interpretation continues to be a rate-limiting step in systems-scale profiling, highlighting the urgent need for innovative approaches capable of comprehensively interpreting and integrating the complex, multi-layered information presented in biological datasets.

Advanced Large Language Models (LLMs), such as OpenAI's GPT-4 and Anthropic's Claude, have proven instrumental in harnessing vast amounts of information embedded in biomedical texts. Prior work has utilized LLMs for knowledge-driven gene prioritization and selection, illustrating this capability. The process involved complex tasks such as identifying functional convergences, scoring candidate genes based on biological and clinical relevance, and fact-checking justifications—all with minimal human intervention. This prior work highlighted the remarkable efficiency of LLMs, in processing and synthesizing biomedical information, thereby facilitating a more informed and rapid selection of candidate genes.

Identifying promising therapeutic targets from thousands of genes in transcriptomic studies remains a major bottleneck in biomedical research. While LLMs show potential for gene identification and prioritization, they suffer from hallucination and lack systematic validation against expert knowledge.

### SUMMARY

Example embodiments relate to a two-stage computational framework that combines LLM-based screening with literature validation for systematic gene prioritization. Starting with 10,824 genes from the BloodGen3 repertoire, multi-criteria evaluation was applied for sepsis relevance, followed by retrieval-augmented generation (RAG) using 6,346 curated sepsis publications. A novel faithfulness evaluation system verified that LLM predictions aligned with retrieved literature evidence.

The present disclosure explores the potential of LLMs in unraveling functional associations within sets of co-expressed genes, pushing the boundaries of context-sensitive analysis in gene expression data. The present disclosure also explores the capabilities of LLMs for elucidating functional connections within co-expressed gene clusters. This exploration aims to advance the scope of context-aware analysis in gene expression data, leveraging the nuanced comprehension of LLMs to reveal deeper biological insights and interactions that conventional methods may overlook.

The framework identified 609 sepsis-relevant genes with >94% filtering efficiency, demonstrating strong enrichment for inflammatory pathways including TNF-α signaling, complement activation, and interferon responses. Literature validation yielded 30 ultra-high confidence therapeutic candidates, including both established sepsis genes (IL10, TREM1, S100A9, NLRP3) and novel targets warranting investigation. Benchmark validation against expert-curated databases achieved 71.2% recall, with systematic correlation between computational confidence and evidence quality. The final candidate set balanced discovery (11 novel genes) with validation (19 known genes), maintaining biological coherence throughout the filtering process.

This framework demonstrates that rigorous methodology can transform unreliable LLM outputs into systematically validated biological insights. By combining computational efficiency with literature grounding, the approach provides a practical tool for prioritizing experimental validation efforts. The modular design enables adaptation to other diseases through knowledge base substitution, offering a systematic approach to literature-guided biomarker discovery.

In a first aspect, a method for automated candidate gene prioritization is provided. The method includes generating a plurality of prompts corresponding to a plurality of candidate genes. Each prompt of the plurality of prompts comprises a predefined scoring criteria to be applied to a corresponding candidate gene. The method also includes selecting a selected prompt from among the plurality of prompts. The method yet further includes prompting a language model with the selected prompt to generate an output. The method additionally includes extracting, from the output, gene-specific information about the corresponding candidate gene. The gene-specific information includes an official name of the candidate gene, a function summary of the candidate gene, evaluative comments regarding each criterion of the predefined scoring criteria, at least one score indicative of the corresponding gene's potential as a biomarker or therapeutic target. The method yet further includes generating a structured database including the gene-specific information for each candidate gene of the plurality of candidate genes. The structured database prioritizes each candidate gene of the plurality of candidate genes based on the corresponding at least one score.

In a second aspect, a method is provided. The method includes providing a set of candidate genes and retrieving, from a trained language model, one or more immunological functions associated with at least one associated gene of the set of candidate genes. The method also includes determining an association score for each of the immunological functions corresponding to each associated gene of the set of candidate genes. The method yet further includes organizing the immunological functions, associated genes, and association scores into a structured CSV file. The method additionally includes determining, for each associated gene, an aggregate association score for an immunological function of interest. The method also includes generating a report for each immunological function.

In a third aspect, a method is provided. The method includes, during a first phase, prompting a language model with a plurality of prompts corresponding to a plurality of candidate genes to generate a set of initial outputs. Each prompt of the plurality of prompts includes a predefined scoring criteria to be applied to a corresponding candidate gene. The method also includes extracting, from the set of initial outputs, a set of initial scores indicative of each corresponding candidate gene's potential as a biomarker or therapeutic target. The method additionally includes, during a second phase, determining, for each candidate gene, a set of relevant documents from a curated document library. The method also includes retrieving, from the curated document library, the set of relevant documents for each candidate gene. Yet further, the method includes prompting a further language model with a further plurality of prompts corresponding to the plurality of candidate genes to generate a set of secondary outputs. The method includes that the further plurality of prompts includes the set of relevant documents for each candidate gene as source documentation. The method also includes extracting from the secondary outputs, a set of secondary scores indicative of each corresponding candidate gene's potential as a biomarker or therapeutic target. The method yet further includes, during a third phase, determining for each candidate gene, based on a comparison between the corresponding initial and secondary outputs, at least one of: a decision classification, a recalibrated score, and a detailed scientific explanation. The method also includes determining, based on the comparison, a final candidate set. The method yet further includes conducting a multi-dimensional optimization analysis on each candidate gene of the final candidate set.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE FIGURES

FIG. 1 illustrates a Computational Framework for Sepsis Gene Prioritization: Multi-Stage LLM-Based Evaluation with Literature Validation and Multi-Dimensional Optimization, according to example embodiments.

FIG. 2A illustrates normalized specificity scores for successfully identified versus missed genes across CTD and DisGeNET databases, according to example embodiments.

FIG. 2B illustrates weighted scores across all 52 benchmark genes for each evaluation method compared to PubMed popularity, according to example embodiments.

FIG. 2C illustrates Top-K overlap performance comparing method rankings against DisGeNET (left) and CTD (right) gene priorities, according to example embodiments.

FIG. 3A illustrates gene-specific weighted score distribution from naive LLM evaluation across 10,824 BloodGen3 genes, according to example embodiments.

FIG. 3B illustrates Scoring heatmap for PS1 genes across eight sepsis-related evaluation criteria, according to example embodiments.

FIG. 3C illustrates functional enrichment analysis comparing PS1 genes against MSigDB Hallmark pathways, according to example embodiments.

FIG. 3D illustrates quantile-specific pathway enrichment heatmap showing functional stratification within PS1, according to example embodiments.

FIG. 4A illustrates RAG evaluation performance across 4,872 gene-query instances, according to example embodiments.

FIG. 4B illustrates gene recovery rates across PS1 quantile clusters following RAG evaluation, according to example embodiments.

FIG. 4C illustrates method agreement analysis comparing evaluation approaches, according to example embodiments.

FIG. 4D illustrates Sankey diagram visualizing cluster membership dynamics between NaiveLLM (PS1, left) and HybridLLM (PS2, right) approaches, according to example embodiments.

FIG. 4E illustrates MSigDB Hallmark pathway enrichment comparison across Evidence set (n=1,279), PS1 (n=609), and PS2 (n=442), according to example embodiments.

FIG. 4F illustrates BG3 module enrichment analysis for PS2 genes showing cell-type specific signatures, according to example embodiments.

FIG. 5A illustrates cluster-specific functional enrichment analysis for PS2's four gene clusters using MSigDB Hallmark pathways, according to example embodiments.

FIG. 5B illustrates principal component analysis (PCA) of PS3 genes (n=82) based on HybridLLM scores across eight evaluation criteria, according to example embodiments.

FIG. 5C illustrates polar plot comparison of mean HybridLLM scores across eight evaluation dimensions for the four PCA-derived clusters, according to example embodiments.

FIG. 5D illustrates comprehensive scoring heatmap for candidate genes (n=30) selected from PCA Cluster 1, according to example embodiments.

FIG. 6 illustrates a method, according to example embodiments.

FIG. 7 illustrates a method, according to example embodiments.

FIG. 8 illustrates a method, according to example embodiments.

### DETAILED DESCRIPTION

Example methods and systems are described herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the figures should not be viewed as limiting. It should be understood that other embodiments might include more or less of each element shown in a given figure. In addition, some of the illustrated elements may be combined or omitted. Similarly, an example embodiment may include elements that are not illustrated in the figures.

### I. OVERVIEW

The following examples describe various methods for utilizing large language models to manipulate, analyze, and profile genetic information for various genomic and bioinformatic applications. In some examples, the biological function of certain genes (e.g., DNA repair, cell cycle, or immune response) can be identified and/or grouped. Once biological functions are identified, pathway analysis and functional annotation can be performed to map experimental data with observed biological features.

### II. (2024-014 PROVISIONAL) (IDF 2024-014) AUTOMATED CANDIDATE GENE PRIORITIZATION WORKFLOW USING LARGE LANGUAGE MODELS

The present disclosure describes an approach for comprehensive functional profiling of gene sets using Large Language Models (LLMs). In some examples, the LLMs could include OpenAI's GPT-4, however other types of LLMs are possible and contemplated.

An example method addresses limitations of traditional functional annotation tools by providing deeper and more context-aware insights into gene functions and interactions. Key aspects of the method include: 1) A stepwise LLM prompting strategy that systematically retrieves, consolidates, and scores immune functions associated with each gene in a set; 2) Generation of ranked lists of functions with detailed justifications and supporting literature evidence; 3) Identification of cell types and transcriptional programs driving the functions associated with the gene set; 4) Integration of LLM outputs with manual curation and fact-checking to ensure accuracy; and 5) An interactive visualization tool for exploring functional associations. The method was demonstrated using Module M10.4 from the BloodGen3 transcriptional module repertoire, revealing a complex network of immune functions beyond what traditional tools typically identify. This approach offers a more granular and quantitative assessment of gene set functional associations, accounting for cellular composition changes and transcriptional regulation. This disclosure establishes a foundation for automated, high-throughput functional profiling of largescale transcriptional module repertoires, potentially facilitating novel biological insights and therapeutic target discovery in various fields of biomedical research.

In various embodiments, an automated workflow is described that leverages large language models (LLMs), specifically GPT-4, to efficiently prioritize candidate genes for targeted assay development. The key aspects include: Python scripts that automate communication with GPT-4 via OpenAI and Microsoft Azure APIs. The scripts handle prompt generation, API requests, response processing, and data storage. In some examples, customizable prompts can be designed to evaluate candidate genes based on criteria such as association with biological processes, biomarker potential, and therapeutic implications. The prompts can be tailored for specific diseases or processes. In some examples, a scoring system is provided that quantifies the evidence supporting each criterion on a scale from 0 to 10, enabling systematic prioritization of candidate genes. In example embodiments, benchmarking experiments can be identified to provide high consistency and reproducibility between the automated API-based approach and manual prompting, validating the effectiveness of the workflow. Examples demonstrate the scalability of the automated approach, successfully prioritizing genes relevant to sepsis from the entire BloodGen3 repertoire comprising 11,465 genes across 382 modules. Examples also demonstrate the adaptability of the workflow to various biological contexts and disease states by modifying the prompts to incorporate disease-specific criteria. The automated candidate gene prioritization workflow offers a more efficient and systematic way to identify promising candidates for targeted assay development from large-scale gene expression datasets. Furthermore, methods described herein have potential applications in biomarker discovery, drug target identification, and the development of targeted diagnostic and prognostic assays for various diseases.

Candidate gene prioritization plays a crucial role in identifying potential biomarkers from large-scale molecular profiling data. Systems-scale profiling technologies, such as transcriptomics, have revolutionized biomedical research by simultaneously measuring tens of thousands of analytes, leading to significant advances in various medical fields, including oncology, autoimmunity, and infectious diseases. However, translating these findings into actionable clinical insights often requires the identification of relevant analyte panels and the design of targeted profiling assays. Targeted transcriptional profiling assays enable precise, quantitative assessments of the abundance of panels comprising tens to hundreds of transcripts. These assays offer several advantages, including cost-effectiveness, rapid turnaround times, and the ability to process large sample numbers, making them valuable for research and clinical applications.

However, the critical task of selecting relevant candidate genes for inclusion in targeted assays can be challenging, especially when contending with the extensive volumes of biomedical information generated by systems-scale profiling technologies.

Knowledge-driven methods for candidate gene prioritization must efficiently sift through vast amounts of literature to identify the most promising candidates. This process can be lengthy and may lack depth due to the sheer volume of information available for each gene. While resources such as gene ontologies and curated pathways can help, they often provide only superficial information about the genes and may lack context. As a result, there is a pressing need for more efficient and effective methods to assimilate and synthesize the extensive, context-rich information necessary for effective gene curation and analysis.

The introduction of Large Language Models (LLMs) has opened new possibilities for leveraging collective biomedical knowledge in candidate gene prioritization. LLMs, such as GPT-4 (OpenAI), Claude (Anthropic), and PaLM (Google), have demonstrated remarkable capabilities in natural language understanding and generation. These models are trained on vast amounts of text data, allowing them to assimilate and synthesize information from diverse sources, including scientific literature. The potential of LLMs in assisting with candidate gene prioritization has been recently explored. In a proof-of-concept study, we demonstrated the utility of LLMs in a manual candidate gene prioritization workflow. The study focused on prioritizing genes forming a circulating erythroid cell blood transcriptional signature, which was previously associated with respiratory syncytial virus (RSV) disease severity, vaccine response, and elevated transcript abundance in patients with metastatic melanoma. This manual workflow involved several tasks, such as identifying functional convergences among candidate genes, scoring genes based on specific criteria (e.g., relevance to erythropoiesis, potential as a biomarker), and selecting top candidates for further characterization. We benchmarked four LLMs (GPT-3.5, GPT-4, Bard, and Claude) and found that GPT-4 and Claude performed the tasks most satisfactorily. The study highlighted the potential of LLMs in enhancing the efficiency and depth of knowledge-driven candidate gene prioritization, with minimal human input.

Building upon this work, we sought to further streamline the candidate gene prioritization process by developing an automated LLM-enabled workflow. The transition from manual prompting via the chat interface to automated prompting using Application Programming Interfaces (APIs) offers several potential advantages. First, API-based automation enables the efficient generation and submission of prompts for a large number of candidate genes, reducing the time and effort required for manual input. Second, it allows for the seamless integration of the LLM-based prioritization workflow with other computational tools and pipelines, facilitating the development of end-to-end solutions for targeted assay design. Third, the API-based approach provides a more standardized and reproducible way of interacting with LLMs, minimizing the variability introduced by human operators. This automated workflow aims to enable the prioritization of extensive module repertoires, such as BloodGen3, and facilitate the design of disease-specific panels, such as ones we previously designed as part of our COVID-19 and pregnancy work. The integration of LLMs into a partially automated candidate gene prioritization pipeline could thus significantly advance biomedical research by accelerating the translation of systems-scale profiling data into targeted assays for clinical and research applications.

### A. Methods

i. Development of Python Scripts for API Communication

To automate the candidate gene prioritization workflow using Large Language Models (LLMs), we developed Python scripts that facilitate communication with GPT-4 through Application Programming Interfaces (APIs). These scripts enable programmatic interaction with the LLM, allowing for the streamlined submission of prompts and the efficient retrieval of generated responses.

We utilized two different APIs to establish a connection with GPT-4: the OpenAI API and the Microsoft Azure API. For the OpenAI API, we leveraged the openai library in our Python script, which provides a convenient way to authenticate and send requests to the API. By setting up the appropriate authentication credentials and specifying the desired model (GPT-4), we were able to programmatically interact with GPT-4 and obtain its responses to our prompts. Similarly, for the Microsoft Azure API, we used the azure-ai-openai library in our Python script to establish a connection with GPT-4 hosted on the Azure infrastructure. This approach provided us with the flexibility to choose between different API endpoints based on factors such as availability, performance, and organizational preferences. The developed Python scripts encompass the entire workflow of the automated candidate gene prioritization process. The scripts perform key functions such as prompt generation, API communication, response processing, and data storage and organization. By encapsulating these functionalities within the Python scripts, we have created a streamlined and automated workflow for candidate gene prioritization using GPT-4.

ii. Automated Candidate Gene Prioritization

Prompts used for gene scoring and prioritization: To prioritize candidate genes using GPT-4, we designed a set of prompts that elicit specific information about each gene, including its official name, function summary, and scores for various criteria relevant to the gene's potential as a biomarker or therapeutic target. The prompts were carefully crafted to cover essential aspects such as the gene's association with different types of interferon responses, relevance to circulating leukocytes immune biology, current use as a biomarker in clinical settings, potential value as a blood transcriptional biomarker, known drug target status, and therapeutic relevance for diseases involving the immune system. The scoring system was designed to provide a quantitative measure of the evidence supporting each criterion, with scores ranging from 0 to 10. A score of 0 indicates no evidence found, while a score of 10 represents strong evidence. Intermediate scores reflect varying levels of evidence quality and reliability, with 1-3 indicating very limited evidence, 4-6 indicating some evidence that needs validation or is limited to certain conditions, and 7-8 indicating good evidence.

iii. API-Based Automation of the Prompting Process

To automate the candidate gene prioritization process, we integrated the designed prompts into the Python scripts that communicate with GPT-4 via the OpenAI and Microsoft Azure APIs. The scripts automatically generate the prompts for each candidate gene by incorporating the gene symbol and the predefined scoring criteria. The generated prompts are then sent to GPT-4 through the respective API, and the model's responses are received and processed by the scripts. The scripts extract the relevant information from the generated text, including the gene's official name, function summary, evaluative comments for each criterion, and the corresponding scores. The extracted data is then stored in a structured format for further analysis and interpretation. This automated process allows for the efficient prioritization of many candidate genes without the need for manual prompt generation and response handling.

iv. Benchmarking Experiments

Manual prompting: To assess the consistency and reproducibility of the gene prioritization results, we conducted benchmarking experiments comparing manual and automated prompting approaches. For manual prompting, we selected three different sites around the world: the United States, Thailand, and Qatar. At each site, a researcher manually submitted the same set of prompts to GPT-4 using the OpenAI web interface. The prompts were submitted in triplicate to evaluate the consistency of the model's responses. The manual prompting process involved copying and pasting the predefined prompts into the GPT-4 interface, replacing the placeholder gene symbol with the actual gene of interest. The researchers then recorded the model's responses, including the gene's official name, function summary, evaluative comments, and scores for each criterion.

API-based automated prompting: For the API-based automated prompting, we utilized the Python scripts developed for automating the candidate gene prioritization workflow. The scripts were configured to generate prompts for the same set of genes used in the manual prompting experiments. The automated prompts were submitted to GPT-4 via the OpenAI API, and the model's responses were automatically processed and stored by the scripts. To assess the consistency and scalability of the automated approach, we performed the automated prompting experiments in triplicate and also increased the number of replicates to five. This allowed us to evaluate the reproducibility of the results and the potential for handling a larger number of candidate genes.

Specific prompt used for benchmarking: For the benchmarking experiments, we used a specific prompt that assessed the gene's relevance to various aspects of immune biology and its potential as a biomarker or therapeutic target, as detailed above and in supplementary methods.

v. Application of the Automated Candidate Gene Prioritization Workflow for Sepsis Monitoring

Selection of candidate genes for prioritization: To demonstrate the scalability and potential clinical application of our automated candidate gene prioritization approach, we focused on developing a targeted assay for monitoring patients with sepsis. We selected the entire BloodGen3 repertoire, consisting of 382 modules, as the candidate gene pool for this use case.

Customization of prompts for sepsis-specific prioritization: We customized the prompt used in the automated prioritization workflow to focus on sepsis-specific criteria. The modified prompt assessed each gene's relevance to sepsis pathogenesis, host immune response, organ dysfunction, biomarker potential, and therapeutic implications. The customized prompt was designed as follows:

For the Gene [input=official Gene Symbol]
1. Provide the gene's official name
2. Provide a brief summary of the gene's function.
3. Give each of the following statements a score from 0 to 10, with 0 indicating no evidence and 10 indicating very strong evidence:
a. The gene is associated with the pathogenesis of sepsis. Score: Based on evidence of the gene's involvement in the biological processes underlying sepsis, including but not limited to its role in the dysregulated host response to infection, organ dysfunction, or sepsis related complications.
b. The gene is associated with the host immune response in sepsis. Score: Based on evidence of the gene's involvement in the immune response during sepsis, including but not limited to its role in innate or adaptive immunity, inflammation, or immunosuppression.
c. The gene is associated with sepsis-related organ dysfunction. Score: Based on evidence of the gene's involvement in the development or progression of organ dysfunction in sepsis, including but not limited to its role in cardiovascular, respiratory, renal, hepatic, or neurological dysfunction.
d. The gene is relevant to circulating leukocytes immune biology in sepsis. Score: Based on evidence linking the gene to the development, function, or regulation of circulating leukocytes in the context of sepsis, including impacts on leukocyte differentiation, activation, signaling, or effector functions.
e. The gene or its products are currently being used as a biomarker for sepsis in clinical settings. Score: Based on evidence of the gene or its products' application as biomarkers for diagnosis, prognosis, or monitoring of sepsis in clinical settings, with a focus on their validated use and acceptance in medical practice.
f. The gene has potential value as a blood transcriptional biomarker for sepsis. Score: Based on evidence supporting the gene's expression patterns in blood cells as reflective of sepsis or its severity, considering both current research findings and potential for future clinical utility.
g. The gene is a known drug target for sepsis treatment. Score: Based on evidence of the gene or its encoded protein serving as a target for therapeutic intervention in sepsis, including approved drugs targeting this gene, compounds in clinical trials, or promising preclinical studies.
h. The gene is therapeutically relevant for managing sepsis or its complications. Score: Based on evidence linking the gene to the management or treatment of sepsis or its associated complications, including its role as a potential target for adjunctive therapies or personalized treatment strategies.

### Scoring Criteria:

0—No evidence found 1-3—Very limited evidence 4-6—Some evidence, but needs validation or is limited to certain conditions 7-8—Good evidence 9-10—Strong evidence

This prompt was integrated into the Python scripts that communicate with GPT-4 via the OpenAI and Microsoft Azure APIs, ensuring that the prioritization process was tailored to the specific requirements of sepsis monitoring.

Execution of the automated prioritization workflow: We executed the automated candidate gene prioritization workflow using the customized prompts for sepsis monitoring. The Python scripts generated prompts for each candidate gene, incorporating the gene symbol and the predefined sepsis-specific criteria. The scripts then communicated with GPT-4 via the APIs, sending the prompts and receiving the model's responses. The scripts extracted the relevant information from the generated text, including the gene's official name, function summary, evaluative comments, and scores for each criterion.

vi. Utilization of LLMs for Manuscript Preparation

In addition to the development and application of the automated gene prioritization workflow, we also explored the potential of LLMs in assisting with the preparation of this manuscript. Specifically, Claude (Anthropic) was utilized for this task. A prior published manuscript was loaded as context, along with relevant email conversations between the authors, data, figures, and key findings from the current study. Claude was employed in an iterative process to generate text from outlines and following general instructions. This process involved multiple rounds of corrections at different levels (section, paragraph, sentence) as needed. The AI assistant was also used for editing and refining the content to ensure clarity, coherence, and adherence to scientific writing conventions. All AI-generated text was reviewed and validated by the human authors, who provided additional context, corrections, and interpretations as needed. The use of LLMs in this manner aimed to streamline the manuscript preparation process while maintaining the accuracy, reliability, and integrity of the presented information through human oversight and validation.

### B. Results

i. Successful Automation of the Candidate Gene Prioritization Workflow

The developed Python scripts successfully automated the candidate gene prioritization workflow using GPT-4 via the OpenAI and Microsoft Azure APIs. The scripts were able to generate the necessary prompts based on predefined templates and input parameters, including constructing prompts for gene scoring and prioritization, and incorporating specific gene symbols and other relevant information.

The scripts efficiently handled the communication with the chosen API by sending the generated prompts to GPT-4 and retrieving the model's responses. The returned data was processed and the relevant information, such as the gene's official name, function summary, evaluative comments, and scores for each criterion, was extracted from the generated text.

The extracted information was then stored in a structured format using Pandas DataFrames and stored in csv format for future use, facilitating further analysis and processing. The scripts organized the data in a way that streamlined the subsequent prioritization and interpretation of the results.

Overall, the developed Python scripts successfully abstracted away the complexities of API communication and data handling, allowing researchers to focus on other aspects of the prioritization and assay development process. The automation of the workflow using GPT-4 via the OpenAI and Microsoft Azure APIs demonstrated the feasibility and efficiency of integrating LLMs into the candidate gene prioritization pipeline.

### Effective Prioritization of Candidate Genes in Module M10.1 Using GPT-4

Our testing focused on module M10.1, an interferon (IFN) response-associated module from the BloodGen3 repertoire. This module is one of six modules belonging to the A28 aggregate, all of which are functionally associated with IFN responses.

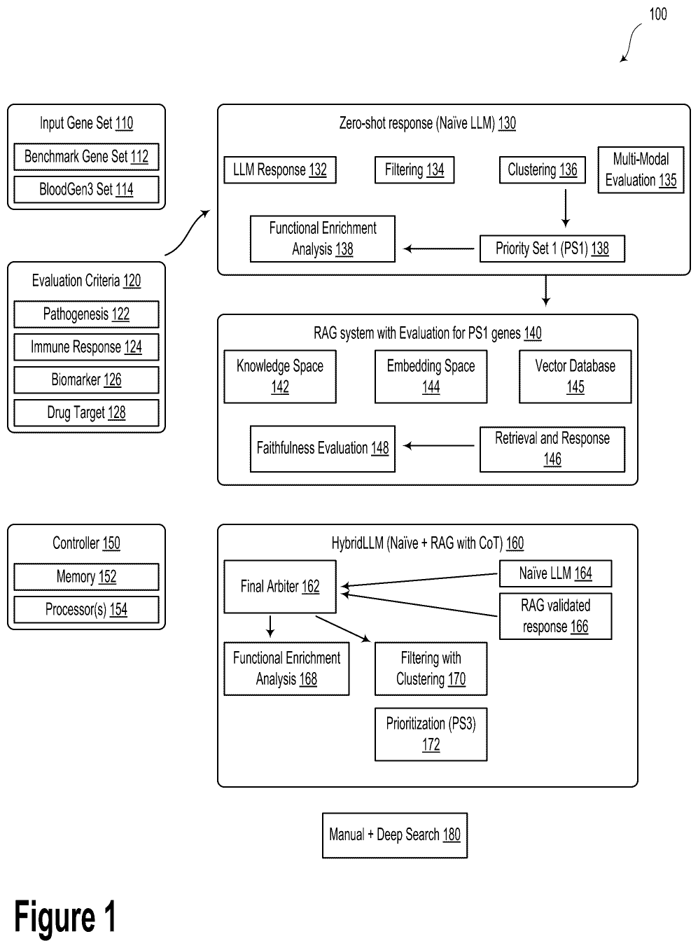

The automated candidate gene prioritization process using GPT-4 and the designed prompts effectively generated informative and structured data for each gene in module M10.1. GPT-4 successfully provided the official gene name, a concise function summary, and evaluative comments for each specified criterion, allowing for a quantitative assessment of the evidence supporting a gene's relevance to IFN responses, therapeutic potential, and other key aspects. FIG. 1 showcases a typical output generated by our automation script, which includes the top 5 genes from module M10.1 and their associated scores across six fields. The genes were selected based on their total score, calculated as the sum of the scores in each field. This structured output enables easy analysis and interpretation of the prioritization results.

Overall, the automated candidate gene prioritization process using GPT-4 effectively integrated LLMs into the workflow, generating informative and structured data for each gene in module M10.1. This approach enabled efficient prioritization based on the genes' relevance to IFN responses and other key criteria, demonstrating the potential of LLMs in streamlining the gene prioritization process.

### Comparison of Manual and Automated Prompting Results

To validate the effectiveness and reliability of our API-based automated prompting approach, we conducted benchmarking experiments comparing its performance with the manual prompting method used in our previous study. These experiments aimed to assess the consistency and reproducibility of the gene prioritization results obtained using the two approaches and to evaluate the scalability of the automated method when dealing with a larger number of candidate genes.

The benchmarking experiments comparing manual and automated prompting approaches demonstrated reasonably high consistency and reproducibility in the gene prioritization results. Correlation analyses of the scores generated by GPT-4 revealed Pearson correlation coefficient values greater than 0.7 across all comparisons, indicating a strong positive relationship between the results obtained through different methods and sites. Higher levels of correlation were observed within each approach, with coefficient values exceeding 0.8 for manual prompting experiments conducted at different sites (United States, Thailand, and Qatar) and values surpassing 0.9 for API-based automated prompting experiments performed with three and five replicates.

These findings suggest that both manual and automated approaches yield consistent results when conducted in a controlled manner. Interestingly, the results generated manually in Qatar showed a higher level of correlation (coefficient>0.85) with the results generated automatically (API) compared to the manual prompting results from the United States and Thailand. This observation suggests that factors other than the prompting approach itself may influence the consistency of the results. Further investigation into the specific factors contributing to this difference is warranted.

Heatmap visualizations reveal lower correlations for individual statements “e” (used as a clinical biomarker) and “g” (known as a drug target), suggesting potential variability in the evaluation of these specific criteria across different prompting approaches.

This variability may be attributed to the complexity and specificity of the information required to assess these statements accurately. Scatter plots comparing gene prioritization scores obtained using the API-based automated approach with 3 replicates (API_3x) against scores from the API-based approach with 5 replicates (API_5x), manual prompting in the United States (Manual_US), and manual prompting in Qatar (Manual_Qatar) were plotted. The high R-squared values indicate strong concordance between the scores generated by the automated and manual approaches, further supporting the consistency and reproducibility of the prioritization results.

The API-based automation of the candidate gene prioritization workflow using GPT-4 offered several advantages over the manual prompting approach, including increased efficiency, reduced human error, and improved scalability. The automated scripts streamlined the process by efficiently generating prompts, extracting relevant information, and organizing the data into a structured format. The benchmarking experiments validated the effectiveness, reliability, and scalability of the API-based automated approach, highlighting its potential for application in larger-scale prioritization tasks. The strong correlations observed between the results obtained through manual and automated approaches, as well as across different sites, underscore the robustness and potential for widespread adoption of this methodology in the field of gene prioritization.

ii. Scalability of the Automated Candidate Gene Prioritization Approach and its Application in Developing a Targeted Assay for Monitoring Sepsis

To demonstrate the scalability of our automated candidate gene prioritization approach, we performed a prioritization run across the entire BloodGen3 repertoire, which consists of 382 modules encompassing 11462 genes. After removing hypothetical and redundant genes, 10,824 genes were analyzed for response. As a use case, we focused on developing a disease-specific targeted assay for monitoring patients with sepsis, a life-threatening organ dysfunction caused by a dysregulated host response to infection.

Early detection and intervention are crucial for improving patient outcomes, and developing a targeted assay for sepsis could aid in the rapid identification of high-risk patients and guide personalized treatment strategies.

Our automated prioritization approach successfully identified sepsis-associated genes across the BloodGen3 repertoire, with distinct gene clusters emerging based on their response patterns to the evaluated criteria. The analysis revealed the proportion and distribution of sepsis-associated genes within module aggregates and individual modules, providing valuable insights for the development of targeted assays.

The analysis of the distribution of sepsis-associated genes across the BloodGen3 module aggregates shows a striking concordance with the functional annotations attributed to these aggregates by Altman et al.

The module aggregates with the highest proportions of sepsis-associated genes identified by our automated workflow include A35 (inflammation, neutrophil activation), A28 (interferon responses), A32-A34 (cytokines/chemokines, inflammation, leukocyte activation), and A8 (monocytes). This aligns well with the known pathophysiology of sepsis, which is characterized by a dysregulated host immune response involving excessive inflammation, neutrophil and monocyte activation, and cytokine production. Interferon signaling has also been implicated in the pathogenesis of sepsis.

Conversely, module aggregates associated with adaptive immunity, such as A1-A3 (T cells, B cells) and A17 (antigen presentation), show a lower representation of sepsis-associated genes. This is consistent with the notion that sepsis is primarily driven by innate immune responses, while adaptive immunity may be suppressed in the acute phase of the disease.

The concordance between the distribution of sepsis-associated genes identified by our automated prioritization approach and the known functional associations of the BloodGen3 module aggregates provides a strong biological validation of our findings. It demonstrates the ability of our workflow to capture relevant disease-associated gene signatures and highlights its potential utility in designing targeted assays for sepsis monitoring and management.

Notably, the entire prioritization run, which involved processing all genes in the BloodGen3 repertoire across 8 statements took approximately 3 days to complete and incurred a cost of about $200. This demonstrates the feasibility and cost-effectiveness of applying our automated approach to large-scale gene prioritization tasks.

The results obtained from this prioritization run highlight the potential of our automated workflow to efficiently identify disease-associated genes across extensive gene repertoires. The insights gained from this analysis can guide the development of targeted assays for sepsis monitoring, focusing on the most relevant gene clusters and modules.

### C. Discussion

This study presents the development and benchmarking of an automated workflow for prioritizing candidate genes, utilizing large language models (LLMs) like GPT-4, to enhance the identification of promising genes for targeted assay development. The method involves Python scripts that interact with GPT-4 through OpenAI and Microsoft Azure APIs, automating the creation and submission of prompts and the extraction of relevant information. Customizable prompts were designed to evaluate candidate genes based on criteria such as association with biological processes, biomarker potential, and therapeutic implications, tailored for specific diseases or processes. By incorporating sepsis-specific criteria into the prompts and considering technical requirements for targeted assay development, we demonstrated the flexibility and scalability of our automated candidate gene prioritization approach. This showcased the potential for adapting the workflow to design targeted assays for various diseases, such as sepsis, based on the specific biological and clinical context. To validate the effectiveness and reliability of the API-based automated prompting approach, we conducted benchmarking experiments comparing its performance with the manual prompting method. These experiments aimed to assess the consistency and reproducibility of the gene prioritization results obtained using the two approaches, as well as to evaluate the scalability of the automated method when dealing with a larger number of candidate genes. By demonstrating the comparability of the API-based approach to the previously established manual workflow, we sought to provide a strong foundation for the adoption of automated LLM-based gene prioritization in the design of targeted assays for various biological and clinical applications. The workflow was applied to prioritize genes from the BloodGen3 repertoire, comprising 382 modules, for sepsis assay development.

This study underscores the potential of incorporating LLMs into gene prioritization, offering a more efficient and systematic way to identify candidates for targeted assays, adaptable to various biological and disease contexts.

The application of large language models (LLMs) in biomedical research has recently gained attention, with several studies exploring their potential in various tasks, such as disease gene prioritization. Kim et al. conducted a comprehensive evaluation of five LLMs, including GPT and Llama2 series, for phenotype-driven gene prioritization in rare genetic disorder diagnosis. While their findings revealed that the best-performing LLM, GPT-4, achieved an accuracy of 16.0%, it still lagging behind traditional bioinformatics tools. However, they observed that prediction accuracy increased with the parameter/model size, highlighting the potential for further improvements as LLMs continue to evolve.

In the context of knowledge-driven gene prioritization, several approaches have been proposed that leverage various data sources and algorithms. For example, Emad et al. developed a method called ProGENI that utilizes prior knowledge of protein-protein and genetic interactions, along with gene expression data, to prioritize genes associated with drug response. Their results demonstrated that knowledge-guided prioritization outperformed other methods and provided new insights into the mechanisms of drug resistance.

Another notable approach is the Monarch Initiative, which integrates open data at a global scale to bridge the gap between genetic variations, environmental determinants, and phenotypic outcomes. The Monarch App combines data about genes, phenotypes, and diseases across species, enabling advanced analysis tools for variant prioritization, deep phenotyping, and patient profile-matching. Interestingly, the Monarch Initiative has also explored the integration of LLMs, such as OpenAI's ChatGPT, to increase the reliability of its responses about phenotypic data. While these studies showcase the potential of knowledge-driven gene prioritization, they often rely on curated databases and predefined feature sets. In contrast, our approach leverages the vast knowledge base and natural language understanding capabilities of LLMs to assess the relevance of candidate genes based on a wide range of criteria and the most up-to-date information available in the biomedical literature.

It is important to note that our work focuses on the prioritization aspect of the gene discovery process, and that the final selection of candidate genes still requires additional steps and manual intervention. Due to the large scale of our study, performing manual fact-checking on the scoring justifications, as we have done in previous work, would be highly laborious or even infeasible, which is another notable limitation. Additionally, we observed some inconsistencies in the scoring, which, coupled with the lack of extensive manual validation, suggests that our work serves more as a proof of concept at this stage, exploring the current capabilities of LLMs in this context. It is also worth noting that the final gene selection process should consider other factors, such as gene expression levels and consistency across reference disease cohorts.

Another limitation of our work is that it was demonstrated on a specific use case, focusing on the prioritization of candidate genes for sepsis monitoring. The prompts could however be readily adapted to other disease settings, either manually or using LLMs for prompt engineering.

In conclusion, our study demonstrates the potential of integrating large language models (LLMs) into the candidate gene prioritization process, offering a more efficient and systematic approach to identifying promising candidates for targeted assay development. By leveraging the vast knowledge base and natural language understanding capabilities of LLMs, our automated workflow can assess the relevance of candidate genes based on a wide range of criteria and the most up-to-date information available in the biomedical literature. The flexibility and adaptability of our approach opens new avenues for the design of targeted assays for diverse applications, such as disease monitoring, treatment response assessment, and the identification of novel therapeutic targets. As LLMs continue to evolve and improve, we anticipate that their integration into biomedical research workflows will become increasingly valuable, enabling researchers to navigate the ever-growing body of knowledge more effectively and efficiently. Future work should focus on further validating and refining our approach across different biological contexts and disease states, as well as exploring the integration of additional data sources and analysis techniques to enhance the accuracy and robustness of the prioritization process. Moreover, the development of standardized protocols and best practices for the use of LLMs in biomedical research will be crucial to ensure the reproducibility and comparability of results across studies. The integration of LLMs into the candidate gene prioritization process has significant implications for the development of targeted assays, particularly in the context of large-scale immune monitoring and research in low-resource settings. By streamlining the identification of the most promising candidate genes, our approach can facilitate the design of cost-effective and targeted assays that focus on the most relevant biomarkers for a given disease or biological process.

### III. (2024-016 PROVISIONAL) LEVERAGING LARGE LANGUAGE MODELS (LLMS) FOR CONTEXTUALIZED IDENTIFICATION OF FUNCTIONAL CONVERGENCES AMONG GENE SETS

### A. BloodGen3 Module Repertoire

Detailed accounts of the BloodGen3 repertoire's development have been previously published. In summary, our prior approach involved analyzing a set of 16 reference datasets that included 985 distinct blood transcriptome profiles, each representing different disease and physiological conditions such as infectious and autoimmune diseases, pregnancy, and organ transplant scenarios. We detected co-clustering patterns which provided the foundation for creating a weighted network. Within this network, we were able to pinpoint highly interconnected networks, or ‘modules’, and further organized these modules into larger groups called ‘aggregates’, informed by the transcript abundance patterns noted across the 16 datasets. This approach facilitated a dual-layered dimension reduction, yielding a more manageable number of variables, either at the level of individual modules (382 variables) or at the aggregated module level (38 variables).

i. Large Language Models

OpenAI's GPT-4, the latest iteration in the series of Generative Pre-trained Transformers, represents a significant advancement in natural language processing technologies. GPT-4's core aim is to parse and generate text that is indistinguishable from that authored by humans. It achieves this through sophisticated unsupervised learning algorithms and is trained on an extensive corpus of text from the internet, encompassing a diverse array of linguistic structures and scenarios. This training ensures GPT-4's proficiency across various domains and its ability to respond to a range of context-specific queries. Notably, its training concluded in April 2023, which marks the cutoff for its knowledge base; hence, it does not assimilate new information post that date, and its responses reflect the dataset available up to that point in time.

ii. Direct Prompting of the Models

PROMPT: Identify functional convergences among this set of genes: [BPI, CEACAM6, 101 CEACAM8, CTSG, DEFA1, DEFA3, DEFA4, ELA2, LOC653600, LOC728358, LOC728358, 102 LOC728358, L TF, MPO, and OLFM4]. Output the results as a table, with the first column indicating the functional convergences and the second column indicating the associated genes.

### LLM Stepwise Prompting: In Depth Delineation of Functional Convergences

In-depth assessment of functional convergences among a set of genes was achieved through a stepwise prompting approach.

Step 1: Gene-by-gene retrieval of associated immunological functions, in triplicates

The first step consisted in prompting GPT-4 to retrieve immunological functions for a given gene. To ensure that the list of immunological functions would be comprehensive we ran this prompt in triplicate.

This was carried out in turn for each of the genes belonging to M10.4.

The output obtained for one of the genes can be found in Supplementary Methods. Outputs for all genes from module M10.4 can be explored interactively via a Prezi application.

PROMPT 1: “In an extensive co-expression analysis across 16 human blood transcriptome datasets, we identified 382 transcriptional modules. One gene, [Gene Symbol], showed notable variance in transcript abundance under diverse pathological and physiological conditions. We are concentrating on securing context-aware functional annotations for [Gene Symbol}. For this, identify the immune processes and states [Gene Symbol} is associated with.”

There is no need to elaborate or provide justifications for those at this point, which can simply be provided as a list.

The output should be generated without resorting to an internet search.

### Notes:

Here we specified for the model not to base its output on the results of an internet search as we found the findings to generally be superficial and poorly reproducible.
The prompts are run in triplicates in three separate sessions.

Step 2: Consolidate immunological functions retrieved by the three runs.

Next a prompt is used to consolidate the functions identified by the three independent runs. The complete prompt for OLFM4 and corresponding output can be found in Supplementary Methods.

PROMPT 2: The following prompt was run multiple times:

“In an extensive co-expression analysis across 16 human blood transcriptome datasets, we identified 382 transcriptional modules. One gene, [Gene Symbol} showed notable variance in transcript abundance under diverse pathological and physiological conditions. We are concentrating on securing context-aware functional annotations for [Gene Symbol}. For this, identify the immune processes and states [Gene Symbol} is associated with.”

There is no need to elaborate or provide justifications for those at this point, which can simply be provided as a list.

The output should be generated without resorting to an internet search. Consolidate the output generated from those multiple runs. Please provide a comprehensive and detailed list ensuring that no association is omitted. After compiling the list, please double-check to confirm that all significant associations are included:
[#1 insert the output from PROMPT 1 from the first run, for the gene in question, =OUTPUT1_Rep1[Gene]]
[#2 insert the output from PROMPT 1 from the second run, for the gene in question, =OUTPUT1_Rep2[Gene]]
[#3 insert the output from PROMPT 1 from the third run, for the gene in question, =OUTPUT1_Rep3[Gene]]

### Notes:

Context is provided in PROMPT2 by including the prompt that was used to generate the output that is to be consolidated.
A follow-on prompt might be used to ask the model to verify its output for completeness. However, we have found that the first output is dependable and using this follow-on prompt does not produce substantial improvements.

Step 3: Generate scores for each gene's associated immunological functions.

The next prompt permits to obtain association scores for the immunological functions identified in the previous step for a given gene.

PROMPT 3: Generate a score indicating the strength of the association between OLFM4 and each of the immunological processes, states or functions listed below. Scoring range is 0-10.

Scoring should be generated without calling on analytics or internet searches. A short justification should be provided.

The scoring criteria are as follows:
0—No Evidence Found: No scientific studies or data support any association between this gene and the immunological process in question.
1-3—Very Limited Evidence: Sparse or preliminary evidence suggesting an association. This may include small-scale studies, findings with low statistical significance, or early-stage research that indicates a potential but not well-established link.
4-6—Some Evidence: Moderate evidence from several studies or a few larger studies. Findings are somewhat consistent but may have some contradictions or limitations in study design.
7-8—Good Evidence: Strong and consistent evidence from multiple well-conducted studies, including larger-scale and possibly meta-analyses. Evidence indicates a clear association, though minor uncertainties may still exist.
9-10—Strong Evidence: Overwhelming and consistent evidence from a wide range of studies, including large-scale, high-quality research and comprehensive reviews. The association is well-established and widely accepted in the scientific community.
[insert the output from PROMPT 2, for the gene in question, =OUTPUT 2_[Gene]]

Step 4: Organize the compiled information into a structured CSV file.

After compiling and scoring the associated immune functions for each gene (Steps 1-3), we instructed GPT-4 to parse this information and organize it in a CSV file format. To avoid overwhelming the model, we ran the prompt separately for each gene, iteratively appending the information to a single CSV file. The prompt used was as follows:

“Create a .csv file to organize the information provided below. The table should have three columns. In the first column, list each immunological function associated. In the second column, include the symbol of the associated gene for each function listed. In the third column, provide the association score. The information to be organized is as follows: [provide output from earlier step, 1 gene at a time]”

We manually verified the accuracy of the final output CSV file, ensuring that all immune functions were extracted and that the cumulative scores matched the expected values. This verification process confirmed GPT-4's capability in parsing and organizing the information effectively. To further refine the data, we manually collapsed some of the categories before proceeding to the next step. For example, “inflammation,” “regulation of inflammation,” and “inflammatory processes” were combined into a single category: “inflammation.”

Step 5: Compute composite scores for each immunological function linked to the gene set. Next, we instruct the model to compute a composite score for the immunological functions identified in the previous step. This is achieved by aggregating the association scores attributed to each gene in the module, a process conducted separately for each immunological function associated with the module. The following prompt is used:

“The .csv file is attached which has three columns. The first column lists immunological functions. The second column includes the symbol of the associated gene for each function listed. The third column includes the association score for each function and gene listed.

Focusing on a specific function, [indicate function of interest], list all associated genes, their association scores and compute an aggregate association score, which is the sum of all association scores for this function. For those genes that are listed more than once, use only one score (the average of all the scores for that gene).”

Step 6: Generate reports for each function:

The outputs from steps 6a, 6b, 6c and 6d, which are detailed below, were used to generate a comprehensive report for each immune function individually. This process involved compiling the summary table (step 6a), cell type associations (step 6b), transcriptional program associations (step 6c), functional convergence narrative (step 6d) into a single document for each immune function.

Once a report was completed for a given immune function, the process was repeated for the next immune function associated with at least three genes from the M10.4 gene set (18 in total). This iterative approach allowed for the systematic generation of detailed reports for each relevant immune function, providing a comprehensive understanding of the functional convergences and biological implications of the genes within the M10.4 module.

Step 6a: generate a summary table with gene symbols, immune functions, association scores, and detailed narratives.

For the immune function that was singled out in the previous step, and following the computation of composite scores, we instructed the model to generate a summary table that includes the gene symbol, immune function, association score, and a detailed narrative describing its association with the immune function. The prompt used was as follows:

“Generate a table listing in the first column the gene symbol, in the second column the immune function, in the third column the association score and in a third column a detailed narrative describing its association with the immune function. The narrative should be based on current, peer-reviewed scientific knowledge and include specific roles these genes are known to play in the immune system.”

Step 6b: Identify cell types associated with the gene set in the context of the specific immune function.

To further contextualize the functional associations of the gene set, for the same immune function, we instructed the model to identify cell types that might be most specifically associated with the genes, considering the patterns of transcript abundance in whole blood across various diseases. The prompt used was as follows:

“This set of genes displayed similarities in patterns of transcript abundance in whole blood across a wide range of diseases. This can be explained by relative changes in the abundance of leukocyte populations. Based on the narratives generated above, describe which cell types this set of genes could be most specifically associated with, in the context of [indicate function of interest].

The output should be organized in a table, with the first column indicating the context (indicate function of interest), the second column indicating the nature of the association (cell type), the third column indicating the associated cell types, along with the symbols of genes which are associated with the cell type, and the fourth column including a justification.”

Step 6c: Identify transcriptional programs associated with the gene set in the context of the specific immune function.

To further elucidate the underlying mechanisms driving the coordinated expression of the gene set, we instructed the model to identify transcriptional programs that might be most specifically associated with the genes, considering the patterns of transcript abundance in whole blood across various diseases. The prompt used was as follows:

“This set of genes displayed similarities in patterns of transcript abundance in whole blood across a wide range of diseases. This can be explained by coordinated transcriptional regulation. Based on the narratives generated above, describe which transcriptional programs this set of genes could be most specifically associated with, in the context of [indicate function of interest].

The output should be organized in a table, with the first column indicating the context [indicate function of interest], the second column indicating the nature of the association (transcriptional program), the third column indicating the associated transcriptional programs, along with the symbols of genes which are associated with the transcriptional program, and the fourth column including a justification.”

Step 6d: Generate a narrative describing the functional convergences among the genes in the context of the specific immune function.

To synthesize the information gathered in the previous steps, we instructed the model to generate a narrative describing the functional convergences observed among the genes in the context of the specific immune function of interest. The prompt used was as follows:

“Next, generate a narrative describing the functional convergences observed among these genes in the context of [indicate immune function of interest]. The style should be direct and technical and should avoid overstatements.”

Step 7: Fact-check individual statements and retrieve backing references.

The final step involves fact-checking the narrative generated in the previous step and providing backing references for the statements, as would be expected in a research article. To accomplish this, we utilized the Claude 3 language model, using the following prompt:

“Provide backing references for the statements in this paragraph, where expected in a research article. If no suitable references can be found, please flag and edit out the statements in question”

Claude 3 was tasked with identifying relevant references to support the statements made in the narrative. If no suitable references could be found for a particular statement, Claude 3 was instructed to flag and edit out the statement in question. The references provided by Claude 3 were then manually checked for factuality and adequacy in justifying or supporting the corresponding statements. This process ensures that the narrative is grounded in current scientific knowledge and that any unsupported claims are removed.

Step 8: Generating summary tables using Claude 3

After compiling all 16 reports into a single PDF file, we utilized the Claude 3 language model to generate multiple summary tables, consolidating the findings from the individual reports. The following prompts were used to generate the summary tables:
1. Generating Table 4—Immune Functions Associated with the M 10.4 Gene Set and Their Cumulative Association Scores: “Please generate a table (Table 4) listing all the immune functions investigated in the attached reports, along with their cumulative association scores. The table should have three columns: ‘Immune Function,’ ‘Cumulative Association Score,’ and ‘Associated Genes (Association Score).’ The rows should be ordered in descending order based on the cumulative association scores.”
2. Generating Table 5—Cumulative and Individual Association Scores of Immune Functions for Each Gene in the M10.4 Gene Set: “Please create a table (Table 5) summarizing the total association scores for each gene in the M10.4 gene set, along with the individual immune functions and their respective association scores. The table should have three columns: ‘Gene,’ ‘Total Association Score,’ and ‘Immune Functions (Score).’ The rows should be ordered in descending order based on the total association scores. Within each cell in the ‘Immune Functions (Score)’ column, the immune functions should also be ordered in descending order based on their individual association scores.”
3. Generating Table 6—Immune Functions Associated with the M10.4 Gene Set, Their Justifications, and Backing References: “Please generate a table (Table 6) presenting the immune functions associated with the M10.4 gene set, along with summarized justifications and backing references. The table should have three columns: ‘Immune Function,’ ‘Summarized Justification,’ and ‘Backing References.’ The justifications should be based on the narratives included at the end of the reports, and the backing references should be derived from the references provided in the reports.”
4. Generating Table 7—Cell Type Associations of the M10.4 Gene Set and Their Corresponding Immune Functions: “Please create a table (Table 7) presenting the cell types associated with the genes in the M10.4 set, based on the immune functions identified in the reports. The table should have three columns: ‘Cell Type,’ ‘Associated Immune Functions,’ and ‘Associated Genes.’ The rows should be ordered in descending order according to the number of associated genes.’”
5. Generating Table 8—Transcriptional Programs Associated with the M10.4 Gene Set and Their Corresponding Immune Functions: “Please generate a table (Table 8) presenting the transcriptional programs inferred from the immune functions associated with the M10.4 gene set, as identified in the reports. The table should have three columns: ‘Transcriptional Program,’ ‘Associated Immune Functions,’ and ‘Associated Genes.’ The rows should be ordered in descending order based on the number of associated genes.”

The resulting tables (Tables 4-8) provide a comprehensive overview of the immune functions, cell types, transcriptional programs, and genes associated with the M 10.4 gene set, as well as their respective justifications and backing references.

iii. Interactive Visualization of Functional Profiling Results

An interactive visualization tool was developed using the Prezi presentation platform to complement the LLM-based functional profiling workflow. An interactive circle plot was created with genes arranged in the outer circle and immune functions represented by color-coded nodes in the inner circles. The color-coding indicates the range of the cumulative association scores for each immune function, while the edges connecting the genes to the immune functions represent the individual association scores. Detailed reports for each immune function, generated through the LLM-based workflow, were embedded within the Prezi presentation. These reports contain comprehensive information on the gene-function associations, including supporting literature evidence. Users can access these reports by clicking on the corresponding immune function nodes within the interactive plot. The Prezi platform was chosen for its ability to create and share interactive presentations online. The platform's features allowed for the development of a proof-of-concept user interface that enables users to explore the functional profiling results intuitively. Such a user interface would provide a centralized resource for accessing and navigating the comprehensive information generated by the LLM-based workflow.

iv. Use of LLMs for Streamlining Manuscript Preparation

We explored the use of Claude 3 “Opus” by Anthropic to assist in the preparation of this manuscript. The authors provided Claude 3 with background information, including an initial draft of the manuscript and key findings, to generate content for specific sections such as the results, discussion, and methods. The LLM-generated text was then iteratively refined through a process of author review, additional prompts for targeted improvements, and manual editing to ensure accuracy, clarity, and coherence. Claude 3 also assisted with literature searches and suggesting relevant references based on its knowledge cutoff of August 2023, which were then manually verified by the authors.

### B. Results

i. Functional Resolution of Fixed Transcriptional Module Repertoires as a Use Case

In this proof of concept, our objective was to elucidate functional relationships within a module (a co-expressed gene set), from the BloodGen3 transcriptional module repertoire. This repertoire includes 382 modules identified through co-clustering analysis across 16 patient cohorts, incorporating nearly 1,000 transcriptome profiles. Notably, this repertoire serves as a standardized framework for data analysis and visualization across multiple projects, including those involving datasets not initially used in its development. The use of such a stable framework allows for accumulated insights into the functional significance of its components over time. The repetitive application of these modules across various studies, spanning years, underscores the value in investing efforts to understand their functional relevance thoroughly. This in turn inspired our current work to establish a methodology for in-depth functional profiling using Large Language Models (LLMs), with the hope of being able to offer deeper and more nuanced insights than traditional approaches.

For our initial exploration, we focused on Module M10.4 of the BloodGen3 repertoire, known for its association with neutrophil microbicidal activity, comprising 13 genes: BPI, CEACAM6, CEACAM8, CTSG, DEFA1, DEFA3, DEFA4, ELA2, LOC653600, LOC728358, L TF, MPO, and OLFM4. We evaluated the effectiveness of LLM-enabled workflows against traditional functional profiling tools in identifying functional convergences within this gene set—which is a crucial step for enhancing the utility of fixed, reusable transcriptional module repertoires as platforms for module-level analysis and interpretation.

ii. Identification of Functional Associations Employing Traditional Functional Profiling Tools

We utilized Ingenuity Pathway Analysis (IPA), a widely recognized software tool in the scientific community for the functional profiling of gene lists, to analyze the genes comprising Module M10.4. The IPA results revealed several significant canonical pathways associated with Module M10.4, as depicted in Table 1. Notably, ‘Neutrophil degranulation’ and ‘Neutrophil Extracellular Trap Signaling’ pathways were identified with the highest statistical significance, aligning with the known function of the module related to neutrophil activity. The ‘Airway Pathology in Chronic Obstructive Pulmonary Disease,’ ‘Activation of Matrix Metalloproteinases,’ and ‘Antimicrobial peptides’ pathways were also statistically significant, suggesting additional biological contexts where these genes might play crucial roles.

The top IPA diseases and biofunctions, as shown in Table 2, support these findings, indicating strong associations with ‘Infectious Diseases,’ ‘Organismal Injury and Abnormalities,’ and ‘Respiratory Disease,’ further substantiating the module's involvement in antimicrobial and inflammatory responses. The analysis also highlighted several molecular and cellular functions and physiological system development and functions, with ‘Cell Death and Survival,’ ‘Cell-To-Cell Signaling and Interaction,’ and ‘Hematological System Development and Function’ among the most significantly associated functions, reinforcing the relevance of M10.4 genes to key immunological processes.

iii. Identification of Functional Associations Via Direct Prompting of the LLMs

Following the functional profiling of the M10.4 gene set with IPA, we proceeded with a direct inquiry approach using GPT-4. This method involved a succinct prompt requesting the identification of functional convergences within Module M10.4's gene set: [BPI, CEACAM6, CEACAM8, CTSG, DEFA1, DEFA3, DEFA4, ELA2, LOC653600, LOC728358, L TF, MPO, OLFM4], with results organized in tabular form. GPT-4 adeptly identified functional themes coherent with the neutrophil activity and antimicrobial function previously known for this module, including Role in Innate Immunity (11 genes), Neutrophil Expression (8 genes), Antimicrobial Activity (6 genes), and Inflammatory Process Involvement (2 genes).

The functional associations identified by GPT-4 echoed the significant canonical pathways such as Neutrophil Degranulation and Neutrophil Extracellular Trap Signaling revealed by IPA, underscoring the consistency across both analytical methods. Furthermore, GPT-4's pinpointing of specific gene functions aligns with the IPA's broader disease and biofunction categories, like Infectious Disease and Antimicrobial Response. This comparison highlights GPT-4's ability to complement the traditional IPA results by providing a more granular and targeted functional perspective. Notably, while IPA furnished a general overview, GPT-4's output was remarkable for its specificity, even within broadly defined categories such as Cellular Growth and Proliferation, and Cell Signaling. This specificity is particularly advantageous when delineating the intricate roles of genes within a well-characterized functional context.

iv. Comprehensive Profiling of Immune Functions Associated with the M10.4 Gene Set Using a Stepwise LLM Prompting Strategy

To further enhance the depth and granularity of the functional profiling for the M10.4 gene set, we employed a comprehensive stepwise LLM prompting strategy. A detailed schematic of the LLM-enabled functional profiling workflow, focusing on individual genes and immune functions. This approach aimed to systematically retrieve, consolidate, and score the immune functions associated with each gene in the module, ultimately generating a ranked list of functions based on their cumulative association scores across the gene set.

The stepwise prompting strategy involved gene-specific retrieval of associated immunological functions, consolidation of functions across multiple runs, scoring of gene-function associations, compilation of module-wide functional landscape, generation of composite scores for each function, and the creation of function-specific narratives. The top-ranking immune functions identified through this approach were Inflammation (cumulative score: 72.0), Antimicrobial Activity (70.0), Neutrophil Activation (69.0), and Innate Immunity (68.5), with sixteen immune functions identified in total with cumulative scores>10. Notably, the stepwise LLM prompting strategy provided a more comprehensive and nuanced understanding of the gene set's functional associations compared to the traditional IPA analysis and the direct LLM prompting approach.

The application of the stepwise LLM prompting strategy thus not only confirmed the central role of M10.4 in neutrophil-mediated innate immunity and antimicrobial defense but also revealed a more intricate network of immune functions, ranging from Inflammation and Neutrophil Activation to Wound Healing and Adaptive Immune Response. This comprehensive functional profiling enhances our understanding of the biological significance of the M10.4 gene set and demonstrates the utility of leveraging LLMs for in-depth characterization of transcriptional modules.

v. Justification and Evidence Supporting the Immune Functions Associated with M10.4

To validate the immune functions identified through the LLM-based approach and ensure that our findings are grounded in existing scientific knowledge, we performed a thorough literature review and compiled a summary of the key evidence supporting each function. This step is crucial for establishing the credibility of our results and providing a solid foundation for the subsequent analyses and interpretations.

Notably, we leveraged Claude 3, to aid in the retrieval of relevant references. By providing Claude 3 with the identified immune functions and their associated genes, we were able to efficiently navigate the vast body of scientific literature and identify the most pertinent studies supporting each function. This innovative application of LLMs in the literature review process not only accelerated the discovery of relevant evidence but also ensured a comprehensive coverage of the available knowledge.

To maintain the highest standards of accuracy and reliability, the relevance of each reference retrieved by Claude 3 was then manually fact-checked. This human-in-the-loop approach permitted to validate the appropriateness of the identified studies and extract the key findings that support the immune functions associated with the M10.4 gene set.

The immune functions, their summarized justifications, and the corresponding backing references may be reported. The justifications highlight the key mechanisms and pathways through which the genes in the M10.4 set contribute to each immune function, while the backing references provide the necessary support from peer-reviewed literature. The table serves as a comprehensive resource for understanding the biological basis of the identified functions and demonstrates the robustness of the LLM-based approach in capturing meaningful and validated associations.

By incorporating this additional layer of validation, we aim to strengthen the confidence in our findings and facilitate their integration with existing knowledge in the field. The justifications and evidence not only reinforce the significance of the identified immune functions but also provide valuable context for interpreting the cell type associations and transcriptional programs that underlie these functions. This integrated approach, combining advanced language models with traditional literature-based validation, represents a powerful strategy for unraveling the complex biology of gene sets and transcriptional modules.

vi. Elucidating Cell Type Associations and Transcriptional Programs Underlying the M10.4

### Gene Set's Immune Functions

To further contextualize the immune functions associated with the M 10.4 gene set, we sought to identify the cell types and transcriptional programs that are most likely to be driving these functions. By leveraging the detailed gene-level information generated through the LLM-based approach, we were able to map the genes to their putative cell type associations and infer the transcriptional programs that may be orchestrating their coordinated expression.

The cell types associated with the M10.4 gene set may be ranked by the number of associated genes. Neutrophils emerged as the most prominent cell type, with 10 out of the 13 genes in the set linked to neutrophil-related functions. This finding reinforces the central role of neutrophils in driving the innate immune response, antimicrobial activity, and inflammatory processes associated with the M 10.4 gene set. Various leukocytes and epithelial cells were also identified as important cellular contexts for the expression of these genes, highlighting the involvement of both innate and adaptive immune cells in the gene set's functional profile.

The transcriptional programs inferred from the gene set's immune functions, may be ranked by the number of associated genes. The innate immune response and antimicrobial response programs were found to be the most prominent, aligning with the gene set's strong associations with innate immunity and host defense. Neutrophil activation and inflammatory response programs were also highly represented, underscoring the importance of these processes in the context of the M10.4 gene set. Notably, the identification of transcriptional programs related to cell adhesion and signaling, immune cell regulation, and lymphocyte modulation suggests that the gene set may also play a role in coordinating the interplay between innate and adaptive immune responses.

The elucidation of these cell type associations and transcriptional programs provides a more comprehensive understanding of the biological mechanisms underpinning the M10.4 gene set's immune functions. By identifying the cellular contexts and regulatory pathways that are most relevant to the gene set, we can better appreciate how these genes work in concert to mount effective immune responses. This knowledge can inform future studies aimed at modulating these genes' expression for therapeutic purposes and guide the development of targeted interventions that harness the innate immune system's potential in combating disease. Moreover, the approach demonstrated here can be applied to other gene sets or transcriptional modules to unravel the complex interplay between genes, cells, and regulatory programs in the context of immune function and beyond.

vii. Interactive Visualization of the M10.4 Gene Set's Functional Landscape

The comprehensive functional profiling of the M10.4 gene set using the LLM-based approach revealed a complex network of immune functions associated with the genes in the module. To better visualize and explore these associations, we generated an interactive circle plot that allows for a more intuitive understanding of the functional landscape. The plot was designed to provide a user-friendly interface for accessing the detailed reports and underlying data generated through the LLM-based workflow.

The resulting visualization highlights the intricate relationships between the genes in the M 10.4 module and their associated immune functions. The color-coding of the immune function nodes based on their cumulative association scores enables a quick identification of the most prominent functions within the module. Moreover, the interactive nature of the plot, allows for an in-depth exploration of the functional associations, providing a valuable resource for researchers interested in understanding the biological significance of the M10.4 gene set.

The development of this interactive visualization tool serves as a proof of concept for the potential of combining LLM-based functional profiling with user-friendly visualization techniques. This approach demonstrates how complex functional profiling data can be made more accessible and interpretable, facilitating the exploration of biological insights. With further refinement and adaptation, this methodology could be applied to other gene sets and transcriptional modules, contributing to a better understanding of the functional organization of biological systems.

### C. Discussion

In this study, we introduced an innovative approach for the functional profiling of gene sets using Large Language Models (LLMs). Our primary objective was to address the limitations of traditional functional profiling tools, which often provide only a surface-level understanding of the biological processes associated with a given set of genes. By leveraging the advanced capabilities of LLMs, specifically OpenAI's GPT-4, we aimed to uncover deeper, more nuanced insights into the functional relationships within a gene set, while accounting for the biological context in which the genes operate.

To demonstrate the utility of our approach, we focused on the functional characterization of Module M10.4 from the BloodGen3 transcriptional module repertoire, a well-defined set of co-expressed genes associated with neutrophil microbicidal activity. We employed a stepwise LLM prompting strategy that systematically retrieved, consolidated, and scored the immune functions associated with each gene in the module. This process yielded a ranked list of immune functions, along with detailed justifications and supporting evidence from the literature, providing a comprehensive and biologically meaningful characterization of the gene set. Our findings not only confirmed the central role of Module M 10.4 in neutrophil-mediated innate immunity and antimicrobial defense but also revealed a more intricate network of immune functions, including for instance chemotaxis, gastrointestinal tract defense, adaptive immunity and barrier function, which are often overlooked by traditional functional profiling tools. By mapping the genes to their putative cell type associations and inferring the underlying transcriptional programs, our tailored approach also accounted for the fact that changes in transcript abundance in complex cell mixtures, such as blood, are driven by relative changes in cellular composition and the activation or repression of specific transcriptional programs. This context-aware interpretation of gene expression patterns is crucial for uncovering the true biological significance of transcriptional modules and their associated functions.

The application of LLMs for functional profiling of gene sets is a relatively unexplored area, with most existing studies focusing on the use of traditional bioinformatics tools and databases.

While these conventional approaches have been valuable in advancing our understanding of gene function and biological pathways, they often rely on predefined annotation categories and may miss important context-specific insights. In contrast, our LLM-based approach allows for a more flexible and adaptive exploration of gene function, leveraging the vast knowledge encoded in these models to uncover novel and biologically relevant associations.

Recent studies have begun to investigate the potential of LLMs and deep learning in various aspects of biological research. However, to the best of our knowledge, our current study is the first to apply LLMs specifically for the functional profiling of transcriptional modules in the context of immune function. By demonstrating the ability of LLMs to provide comprehensive and biologically meaningful characterizations of gene sets, our work opens up new avenues for the application of these powerful tools in systems biology and immunology research. Moreover, our approach complements existing methods for the analysis and interpretation of transcriptional module repertoires, such as the BloodGen3 resource used in this study. These repertoires provide a valuable framework for understanding the co-expression patterns and functional relationships among genes across diverse biological conditions. By integrating LLM-based functional profiling with these established resources, we can gain a more comprehensive and nuanced understanding of the biological processes and pathways associated with specific gene sets, ultimately facilitating the discovery of novel therapeutic targets and biomarkers.

Our study demonstrates the potential of LLMs for comprehensive functional profiling of gene sets, but it is important to acknowledge the limitations of our current approach. The main challenge is the lack of automation in the workflow, which requires significant manual intervention. The stepwise prompting strategy, while effective in eliciting detailed and biologically relevant information from the LLMs, is somewhat intricate and time-consuming. This limits the scalability of the approach, particularly when dealing with larger gene sets or more extensive transcriptional module repertoires.

It is worth noting that the manual corrections required during the fact-checking process were primarily focused on addressing inaccuracies in the references, such as incorrect volumes, page numbers, or journals, or instances where the provided reference did not adequately support a statement. Importantly, we did not encounter instances of reference hallucination when using the specific prompts employed in this study. While hallucinations may still occur with other prompts, even when using advanced models like Claude 3 or GPT-4, the issue has become less prevalent compared to a few months ago.

Furthermore, the investment in time required for the manual curation and fact-checking steps may be justified in the context of our work, which focuses on the functional characterization and interpretation of modules that form a fixed, reusable repertoire. As we have previously demonstrated repertoires can serve as a framework for data analysis and interpretation across numerous studies spanning several years. Thus, the upfront effort invested in thoroughly annotating and validating the functional associations of these modules can yield long-term benefits in terms of the robustness and reproducibility of the analyses.

The proof of concept presented in this study focused on the in-depth characterization of a relatively small gene set, specifically the 13 genes comprising Module M10.4, and their associations with immune functions. However, the workflow can be adapted to retrieve associations between genes and other biological concepts, such as molecular pathways, diseases, or drugs, demonstrating its versatility and potential for a wide range of applications. The applicability of this approach to larger gene sets, containing hundreds or even thousands of genes, remains to be determined, and further investigation is warranted to assess its scalability.

The manual curation and fact-checking steps, which are currently crucial for ensuring the accuracy and reliability of the generated information, may become less resource-intensive as the performance of language models continues to improve. In the preliminary work presented in this manuscript, while some errors were identified in the references provided by the models, these did not lead to any major changes in the gene-immune function associations. This observation suggests that the need for extensive manual fact-checking might decrease over time as the models become more reliable and a sufficient level of trust is established. Nevertheless, it is important to maintain a critical eye and regularly validate the outputs of these models until their performance consistently meets the high standards required for scientific research. It is important to note that our work presents a proof-of-concept workflow, demonstrating the feasibility and potential value of using LLMs for functional profiling of gene sets. However, for this approach to become widely applicable and scalable, further development of tools and automation via API integration is essential.

By addressing these limitations and challenges, future studies can build upon the foundation laid by our work, ultimately enabling the high-throughput, automated functional profiling of large-scale transcriptional module repertoires and other gene set collections. This will facilitate the discovery of novel biological insights and the identification of potential therapeutic targets and biomarkers, accelerating the translation of systems-level molecular profiling data into actionable knowledge.

### IV. AUTOMATING CANDIDATE GENE PRIORITIZATION WITH LARGE LANGUAGE MODELS: FROM NAIVE SCORING TO LITERATURE-GROUNDED VALIDATION

### A. Introduction

Candidate gene prioritization plays a crucial role in identifying potential biomarkers from large-scale molecular profiling data. Systems-scale profiling technologies, such as transcriptomics, have revolutionized biomedical research by simultaneously measuring tens of thousands of analytes, leading to significant advances in oncology, autoimmunity, and infectious diseases. However, translating these findings into actionable clinical insights requires identifying relevant analyte panels and designing targeted profiling assays. Targeted transcriptional profiling assays enable precise, quantitative assessments of tens to hundreds of transcripts, offering cost-effectiveness, rapid turnaround times, and high-throughput capability. The critical challenge lies in selecting relevant candidate genes from extensive biomedical information volumes generated by systems-scale profiling technologies.

Traditional knowledge-driven methods face multiple challenges in efficiently processing vast literature to identify promising candidates. While gene ontologies and curated pathways provide valuable information, they rely on static knowledge bases that may not capture current research findings or miss relevant associations buried within literature. Moreover, traditional approaches rely heavily on static knowledge bases that may not capture the most current research findings or may miss relevant associations buried within the literature. Manual curation, though thorough, is time-intensive and may lack comprehensive coverage due to information volume constraints.

Computational approaches have emerged to address these limitations. Methods like ProGENI integrate protein-protein and genetic interactions with expression data for drug response prediction, while the Monarch Initiative combines multi-species data for variant prioritization. However, recent large language model (LLM) applications in biomedical gene prioritization show mixed results. Kim et al. achieved only 16.0% accuracy with GPT-4 for phenotype-driven gene prioritization in rare genetic disorders, lagging traditional bioinformatics tools. These limitations highlight the need for sophisticated approaches addressing fundamental challenges in LLM-based biomedical inference.

LLMs such as GPT-4, Claude, and PaLM have demonstrated remarkable natural language understanding capabilities, trained on vast text data enabling synthesis of diverse information sources including scientific literature. However, naive LLM approaches face significant biomedical limitations: potential hallucinations, reliance on training data snapshots potentially missing current knowledge, and difficulty providing verifiable, literature-grounded justifications.

Recent advances in retrieval-augmented generation (RAG) and chain-of-thought reasoning offer promising solutions. RAG systems dynamically retrieve and incorporate relevant literature, providing current and verifiable evidence for gene prioritization. Combined with faithfulness evaluation techniques verifying model outputs against retrieved evidence, these approaches significantly enhance reliability and interpretability. Chain-of-Thought reasoning enables multi-perspective evaluation and structured synthesis of competing information sources.

We previously demonstrated LLM utility in manual candidate gene prioritization, focusing on circulating erythroid cell blood transcriptional signatures associated with respiratory syncytial virus disease severity, vaccine response, and metastatic melanoma. Benchmarking four LLMs across multiple criteria, GPT-4 and Claude showed superior performance through consistent scoring (correlation coefficients>0.8), strong alignment with manual literature curation, and evidence-based justifications. This established foundations for automated gene prioritization while highlighting needs for systematic validation and evidence verification.

Building upon this work, we developed a comprehensive framework integrating multiple advanced NLP techniques to address naive LLM limitations. Our framework combines RAG using curated biomedical literature, systematic faithfulness evaluation reducing hallucinations, and Chain-of-Thought reasoning for structured evidence synthesis. This multi-layered approach enables literature-grounded gene prioritization with systematic validation against external reference standards and functional biological coherence assessment, addressing key challenges: verifiable evidence grounding, systematic uncertainty handling, and scalable processing while maintaining interpretability.

This integration of advanced NLP techniques into a validated candidate gene prioritization pipeline represents significant methodological advancement beyond routine LLM applications. The framework enables systematic processing of extensive module repertoires like BloodGen3 while providing robust validation through external benchmarking, cross-model comparison, and biological coherence assessment, accelerating translation of systems-scale profiling data into validated, literature-supported targeted assays for clinical and research applications.

### B. Methods

i. Automated Gene Prioritization Framework

We developed a comprehensive workflow accessing large language models for automated gene prioritization using LlamaIndex (v0.12.37) for RAG development. Initial naive LLM runs were conducted using GPT-4T (April 2024) and GPT-4o (April 2025), with GPT-4o subsequently serving as RAG synthesizer and final arbitrator. The framework incorporates retrieval-augmented generation, faithfulness evaluation, and Chain-of-Thought reasoning, with Ollama (v0.4.8) deployment for local access to open-source models (particularly Phi-4 for faithfulness evaluation). Biomedical named entity recognition used SpaCy (v3.7.5). The system handles prompt generation, model communication, response processing, and data storage in an integrated pipeline using Python 3.8 and OpenAI API (v1.75). A flow chart of the pipeline is given in FIG. 1.

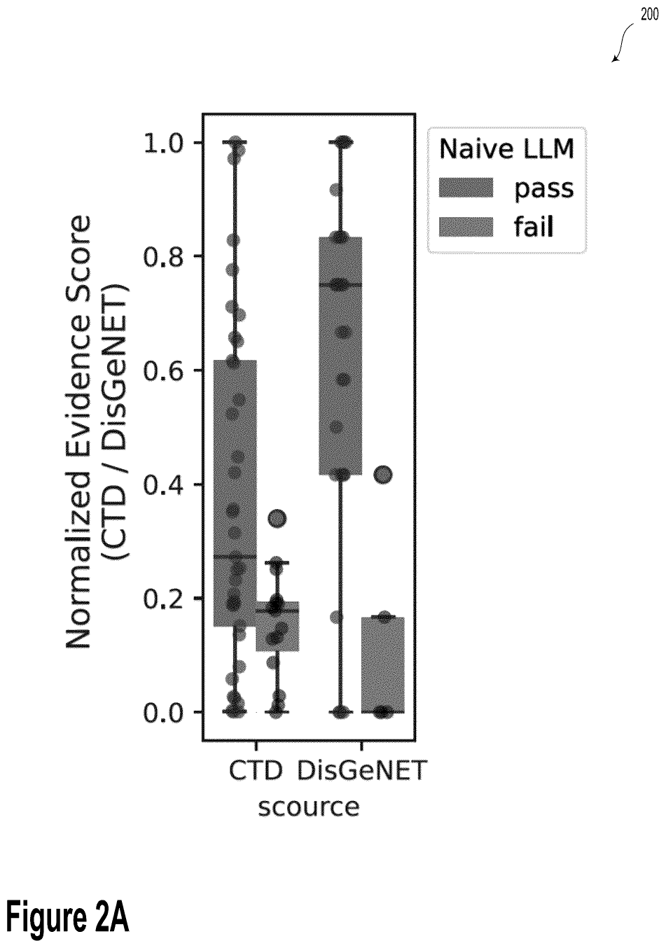

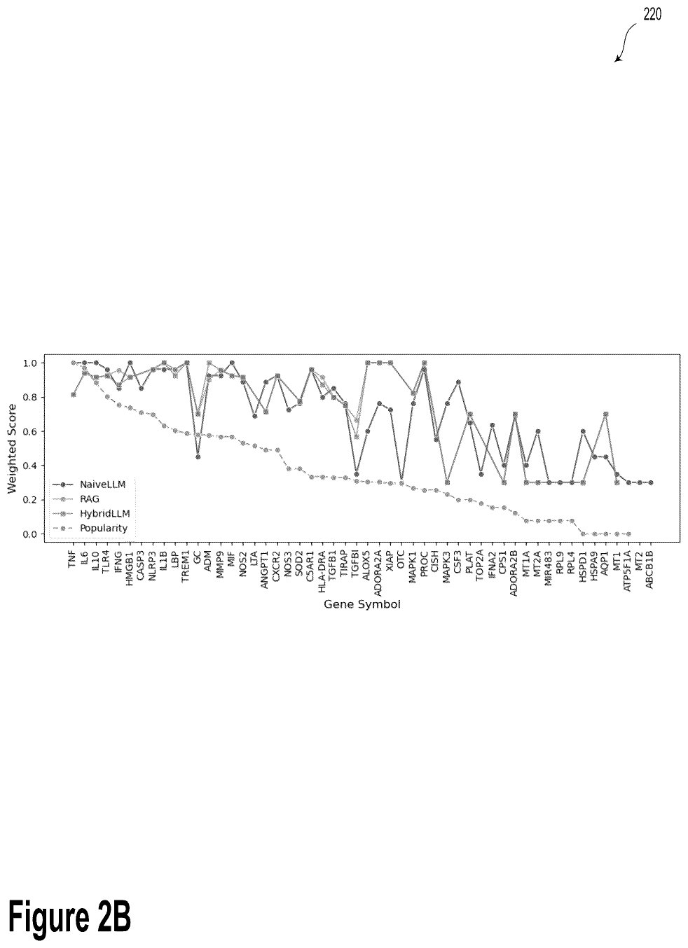

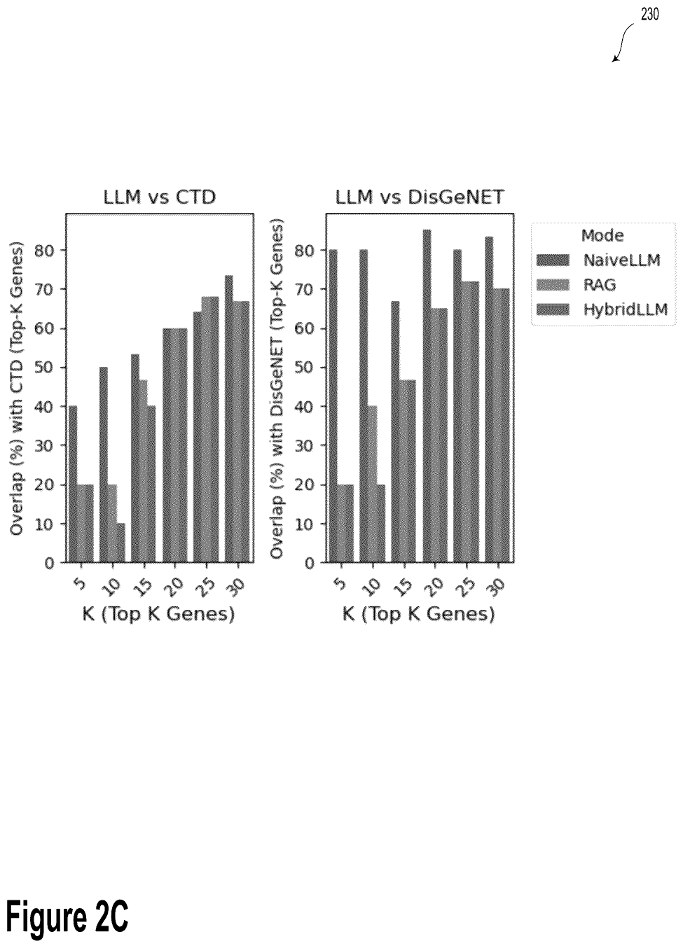

The framework processes input gene sets through progressive filtering and validation stages to identify high-confidence sepsis therapeutic targets. The workflow was first run on the 52-gene sepsis benchmark set (results in FIG. 2) before being applied to the full BloodGen3 compendium. Stage 1: Zero-shot evaluation 130 using Naive LLM across eight sepsis-related criteria (pathogenesis association, immune response, biomarker potential, drug target feasibility) applied to BloodGen3 gene set (>10K genes). Genes meeting scoring thresholds undergo clustering 136 and multi-modal evaluation 135, generating Priority Set 1 138 (PS1, 609 genes). Stage 2 140: RAG-enhanced evaluation system processes PS1 genes through sepsis-specific knowledge base (˜6K literature sources, >10K abstracts) using SPECTER2 embeddings and LlamaIndex framework. Retrieved literature 146 undergoes faithfulness evaluation 148, and RAG-validated responses 166 combine with Naive LLM outputs 164 in HybridLLM approach using chain-of-thought reasoning. Stage 3 160: Literature-validated genes (Priority Set 2, PS2, 442 genes) undergo functional enrichment analysis 168 and multi-dimensional clustering 170. PCA-based optimization identifies top-performing gene cluster, generating ultra-high confidence Priority Set 3 172 (PS3, 82 genes) and candidate set (30 genes). Final Validation 180: PS3 candidates undergo manual curation and deep-search verification to ensure robust evidence support. Color coding distinguishes processing stages: computational evaluation (gray), literature validation (teal), functional optimization (green), and manual verification (blue). The progressive filtering approach reduces 10,824 genes to 30 high-confidence therapeutic targets through systematic computational assessment, literature grounding, and multi-dimensional optimization.

ii. Gene Scoring and Prompt Design

Prompts elicited gene information including official name, function summary, and quantitative scores (0-10 scale) across biomedically relevant criteria. The scoring rubric: 0=no evidence; 1-3=very limited evidence; 4-6=some evidence requiring validation; 7-8=good evidence; 9-10=strong evidence. For sepsis prioritization, we assessed eight criteria: pathogenesis association, host immune response, organ dysfunction, circulating leukocyte biology, clinical biomarker use, blood transcriptional biomarker potential, drug target status, and therapeutic relevance.

iii. RAG Pipeline Implementation

### Knowledge Base Construction

We curated 6,346 sepsis-related documents (4,441 articles, 1,905 reviews, 1990-2025) from NCBI PubMed Open Access, filtered using OpenAlex for high citation percentile (>0.8) (FIG. S1). To enhance coverage, we supplemented this collection with 9,557 abstracts from relevant sepsis publications that were behind paywalls, ensuring comprehensive literature representation across both freely accessible and subscription-based sources. Documents underwent preprocessing (reference removal, chunk segmentation, biomedical NER), SPECTER2 embedding (allenai/specter2_base), and ChromaDB storage with metadata tagging.

iv. Retrieval and Synthesis

For each gene-query instance, we implemented two-stage retrieval: vector similarity search (top_k=25) followed by cross-encoder reranking (SentenceTransformerRerank, top_k=10). Retrieved context was formatted into structured prompts for GPT-4o (temperature=0.1) with explicit source attribution, maintaining query isolation to prevent cross-contamination.

### Faithfulness Evaluation

We conducted comparative faithfulness analysis using two independent evaluators (Phi-4 and GPT-o3-mini) on a representative set of 2,928 gene-query instances from 399 genes (FIG. S2). Each GPT-4o justification received binary classification (“Pass”/“Fail”) from both evaluators. Dual-evaluator agreement reached 71.94% overall with 90.6% agreement in high-confidence cases (Table S1, FIG. S3). Phi-4 was selected as primary evaluator based on: (1) architectural independence from GPT-4o reducing bias, (2) consistent alignment with GPT-o3-mini in strong-evidence cases, (3) lower variance in ambiguous cases, and (4) local deployment via Ollama enabling cost-effective, reproducible evaluation. Only faithfulness-passing instances proceeded to hybrid evaluation.

### Chain-of-Thought Hybrid Reasoning

We developed a hybrid evaluation strategy synthesizing naive LLM knowledge (GPT-4o) with retrieved contextual evidence (RAG) to resolve inference divergences and persistent ambiguities. The Chain-of-Thought framework employed three structured roles: (1) Naive LLM Critic assessing initial predictions for assumptions and overconfidence, (2) Retrieved Evidence Analyst evaluating contextual support quality and specificity, and (3) Final Arbiter synthesizing perspectives with preference for strong retrieved evidence and explicit reasoning for discrepancies. This produced unified outputs: decision classification (High/Medium/Low), recalibrated score (0-10), and detailed scientific explanation.

v. Framework Validation Through Benchmark Analysis

### Benchmark Dataset Selection and Validation Strategy

To establish framework validity before large-scale application, we systematically evaluated performance against curated sepsis gene sets from two complementary databases. DisGeNET (n=32)(26) was selected for mechanistic gene-disease associations derived from systematic literature curation with transparent evidence scoring (ScoreGDA), while CTD (n=48)(27) provided therapeutic and chemical interaction relationships with experimental validation emphasis. These databases were chosen based on methodological transparency, evidence quality standards, and peer-reviewed curation processes (Supplementary Methods: “Benchmarking Strategy and Dataset Selection”). The combined gene sets yielded 52 unique genes with established sepsis associations through expert curation (referred to as the “52-gene sepsis benchmark set”). Additionally, we have compiled a reference gene set (n=929 genes) from other known datasets (like Entrez: DisGeNET, CTD, Monarch, OpenTarget, Sepon and IPA) (FIG. S4). Detail of gene set curation for evaluation is given in Supplementary Methods (section 4).

vi. Gene-Specific Weighted Scoring Calculation

Each gene received scores (0-10 scale) across eight sepsis-related evaluation criteria, as mentioned above (FIG. S5). Individual criterion scores were categorized into three confidence bins: High (≥7), Medium (4-6), and Low (≤3). For each gene, the proportion of scores in each bin was calculated by dividing the count by the total number of criteria (n=8). Weighted scores were computed using confidence-based weights: High×1.0, Medium×0.7, and Low×0.3, with final scores ranging from 0.0 to 1.0. This weighted aggregation was essential to address the inherent unreliability of individual LLM scores by strategically rewarding genes that demonstrated consistent high-confidence evidence across multiple independent evaluation criteria. This approach helps in prioritizing robust multi-dimensional relevance over potentially misleading individual assessment. This methodology was applied consistently across naive LLM, RAG, and hybrid evaluation approaches to enable direct cross-method comparison.

vii. Statistical Analysis and Performance Evaluation

Performance assessment used contingency tables for score transition analysis and classification reports computing precision, recall, and F1-scores. Correlation coefficients were calculated for continuous comparisons with statistical significance testing where appropriate.

### C. Results

Our aim was to convert large-language-model (LLM) outputs into a rigorously validated list of sepsis-relevant genes. To do so, we built a three-stage pipeline that (i) assigns composite scores to every gene with a naïve LLM, (ii) filters those scores through a retrieval-augmented generation (RAG) step with dual-model faithfulness checks, and (iii) refines the surviving genes by multi-dimensional optimization (FIG. 1). We first asked whether this framework could recover genes that expert curators already accept as sepsis-associated before deploying it at genome scale.

i. Benchmark-Based Validation of the Framework

To establish framework validity before large-scale application, we evaluated performance against a 52-gene sepsis benchmark set derived from DisGeNET (n=32 genes) and CTD (n=48 genes) representing mechanistic and therapeutic associations respectively. The framework achieved 71.2% recall (37/52 genes) using our scoring threshold of ≥5 in any evaluation category, with systematic performance variation between databases: DisGeNET 84.4% recall (27/32) and CTD 70.8% recall (34/48) (Table S2). Analysis revealed systematic correlation with expert evidence quality, with identified genes showing significantly higher curation scores than missed genes (FIG. 2A). The three evaluation approaches exhibited distinct but appropriate scoring behaviors (FIG. 2B). All methods demonstrated expected correlations with PubMed publication frequency that mirror expert-curated databases: naive LLM showed strong correlation (Spearman ρ=0.795, p<0.001), while RAG and hybrid approaches showed moderate correlations (ρ=0.528 and ρ=0.524 respectively, both p<0.001) (Table S3). These correlations align with benchmark database patterns (ScoreGDA ρ=0.802, CTD ρ=0.515), confirming that higher scores appropriately reflect genes with more robust evidence bases rather than random scoring behavior. Notably, RAG showed specific enhancements for less-studied genes (AQP1, MIR483), suggesting capability to identify promising but understudied candidates. (Table S2).

Top-K overlap analysis revealed distinct method characteristics for practical applications (FIG. 2C). NaiveLLM demonstrated superior ranking performance with 80% overlap for top-5 DisGeNET genes and 50% for CTD genes, maintaining strong performance through top-30 predictions. In contrast, RAG and HybridLLM showed substantially lower top-K performance, with only ˜20% overlap for top-5 predictions in both databases, improving gradually to 45-70% only at top-30. This pattern reveals a fundamental trade-off: while RAG and hybrid approaches sacrifice immediate ranking performance, they provide literature verification and enhanced detection of understudied candidates.

Systematic analysis identified 15 genes consistently missed across both databases, representing coherent biological categories including protein synthesis (RPL9, RPL4), metabolic regulation (OTC, CPS1, ATP5F1A, GC), cellular maintenance (MT1, MT2, MT1A, AQP1, ABCBIB, HSPA9, TGFBI), and regulatory mechanisms (MIR483, TOP2A). These categories primarily represent housekeeping functions that may be underrepresented in sepsis-specific literature despite biological relevance, highlighting the challenge of identifying functionally important but domain-understudied genes. This validation established framework reliability with characterized blind spots in metabolic pathways, providing performance boundaries essential for confident large-scale application.

ii. Large-Scale Gene Screening and Functional Validation

Building on the validation results, we conducted genome-wide screening of the BloodGen3 repertoire (10,824 genes) to comprehensively identify sepsis-relevant gene candidates across the human transcriptome.

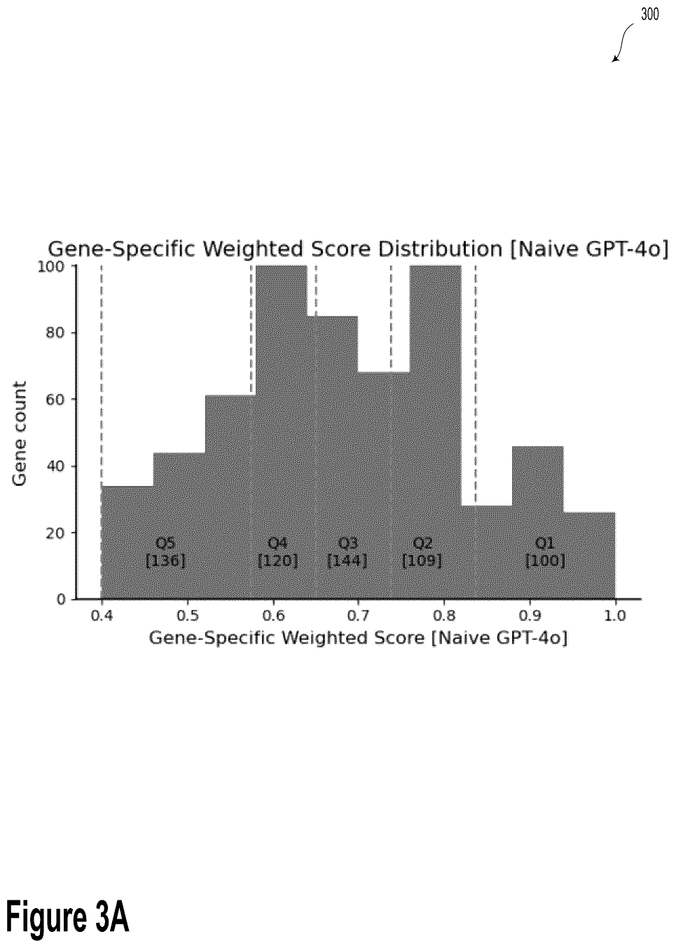

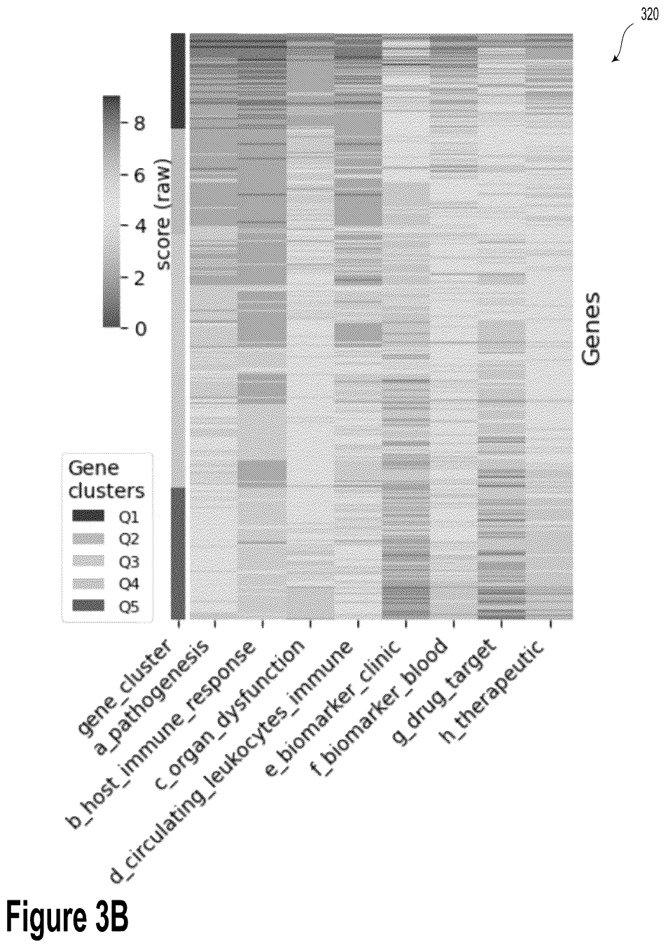

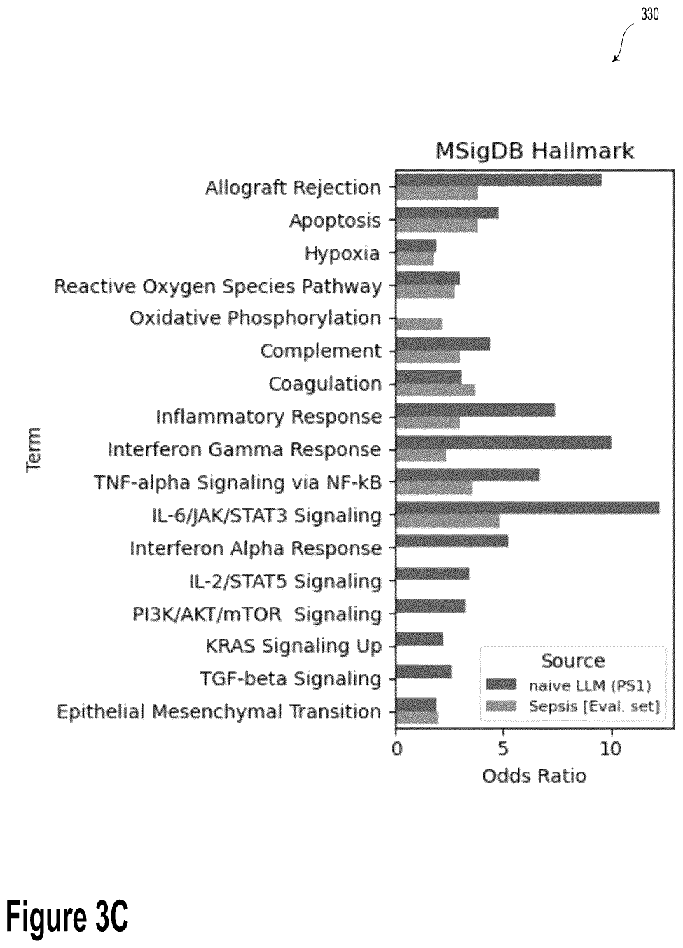

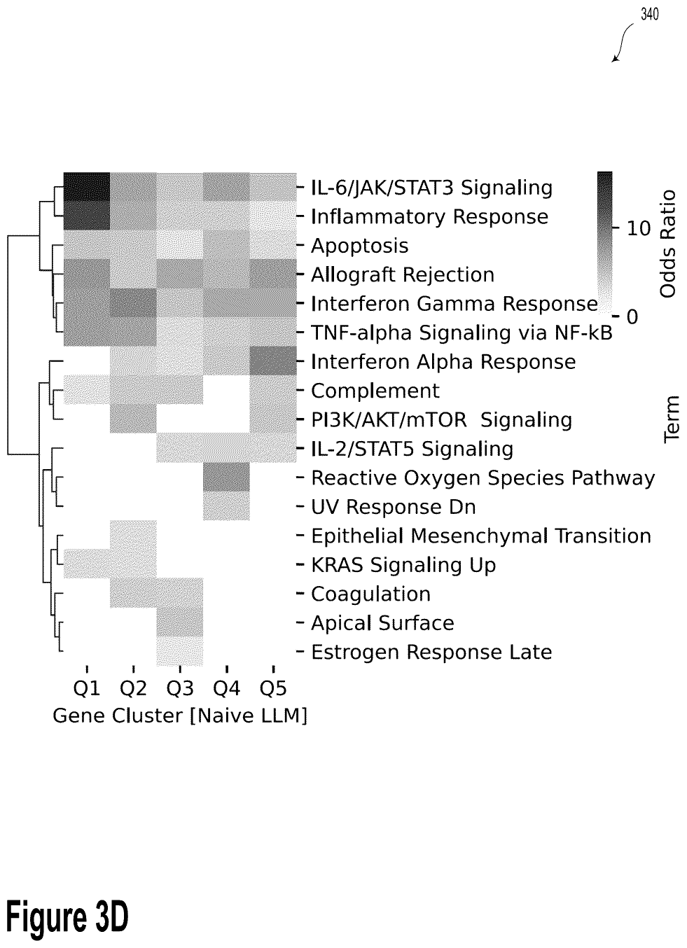

Genome-Wide Filtering and Prioritization. Genome-wide screening using our eight sepsis-related evaluation criteria (pathogenesis association, immune response, clinical biomarker potential, therapeutic relevance, and others) yielded 609 genes with >94% filtering efficiency (FIG. 3A). We designated this filtered set as PS1 (Priority Set 1). To systematically analyze the score distribution, we stratified PS1 genes into five quantile-based groups using naive weighted scores: Q1 (top 100 genes), Q2 (109 genes), Q3 (144 genes), Q4 (120 genes), and Q5 (bottom 136 genes). This quantile-based stratification allows systematic examination of performance gradients across the priority gene set.

Cross-Model Validation. To assess framework robustness across different LLM versions, we conducted comparative analysis between GPT-4o (current model) and GPT-4T (April 2024) on a subset of genes. The cross-model evaluation demonstrated high concordance with 74% agreement in gene prioritization decisions (FIG. S6), indicating that our framework generates consistent results across model iterations and supporting the reliability of our large-scale screening approach.

Score Distribution Patterns Across Quantiles. The scoring heatmap revealed clear performance gradients across quantile groups (FIG. 3B), as expected. Q1 genes (highest-scoring quantile) demonstrated consistently elevated scores across all eight evaluation criteria, representing the most promising sepsis candidates with strong evidence across multiple domains. Q2 genes maintained moderately high scores with some criteria-specific variations. The middle and lower quantiles (Q3-Q5) exhibited progressively more heterogeneous scoring patterns, with Q5 genes showing the most variable performance across criteria, suggesting these represent candidates with more limited or specialized evidence profiles.

Biological Coherence and Functional Stratification Analysis. To demonstrate that PS1 represents a biologically meaningful gene set rather than random selection artifacts, we performed functional enrichment analysis against MSigDB Hallmark pathways (FIG. 3C, Table S4). The PS1 genes showed significant enrichment for pathways directly relevant to sepsis pathobiology, including TNF-α signaling via NF-κB, interferon gamma response, complement activation, inflammatory response, and reactive oxygen species pathways (all FDR<0.05). This enrichment pattern confirms that our computational framework successfully captured genes with coherent biological relevance to the queried sepsis context. As a validation control, we compared PS1 enrichment patterns against the pooled evaluation gene set (929 genes) used in our initial framework development. The PS1 genes demonstrated comparable or enhanced enrichment magnitudes for sepsis-relevant pathways, confirming that the genome-wide screening successfully identified contextually appropriate genes rather than introducing systematic bias.

Score-Based Functional Stratification. Quantile-wise enrichment analysis revealed functional organization that correlates with our scoring hierarchy (FIG. 3D). Higher-scoring quantiles (Q1-Q2) demonstrated stronger enrichment for inflammatory response pathways including IL-6/JAK/STAT3 signaling, complement activation, and TNF-α responses, consistent with their elevated scores across multiple sepsis-related criteria. Mid-tier quantiles (Q3-Q4) showed moderate enrichment for interferon responses and cellular stress pathways, while lower-scoring genes (Q5) exhibited more heterogeneous enrichment patterns including epithelial mesenchymal transition and metabolic processes. This functional gradient supports the validity of our scoring-based prioritization approach, suggesting that higher computational scores correspond to stronger associations with core inflammatory processes relevant to sepsis, while lower-scored genes may represent more peripheral or context-specific mechanisms. Consistent with evidence-based prioritization, PS1 gene clusters showed expected correlation with publication popularity (FIG. S7), where higher-scoring clusters (Q1-Q2) contained genes with greater research attention compared to lower-tier clusters (Q4-Q5), validating that our framework appropriately weights established evidence while maintaining capability to identify less-studied but functionally relevant candidates.

This genome-wide screening successfully identified a functionally coherent set of sepsis candidates while maintaining high specificity for disease-relevant pathways and demonstrating robust cross-model consistency, providing a comprehensive foundation for subsequent detailed analysis.

iii. Literature-Grounded Evaluation Through Retrieval-Augmented Generation

RAG Evaluation Framework and Performance. We implemented a sepsis-specific RAG system incorporating open source literature sources (reviews and research articles) and >10,000 PubMed abstracts using SPECTER 2 and LlamaIndex (see Methods). Each of the 609 PS1 genes was evaluated across eight sepsis-related queries, generating 4,872 total evaluation instances. RAG responses underwent independent faithfulness evaluation using LlamaIndex's Faithfulness evaluator with Phi4 as an independent LLM model, providing binary Pass/Fail assessments based on alignment between retrieved literature chunks and generated justifications.

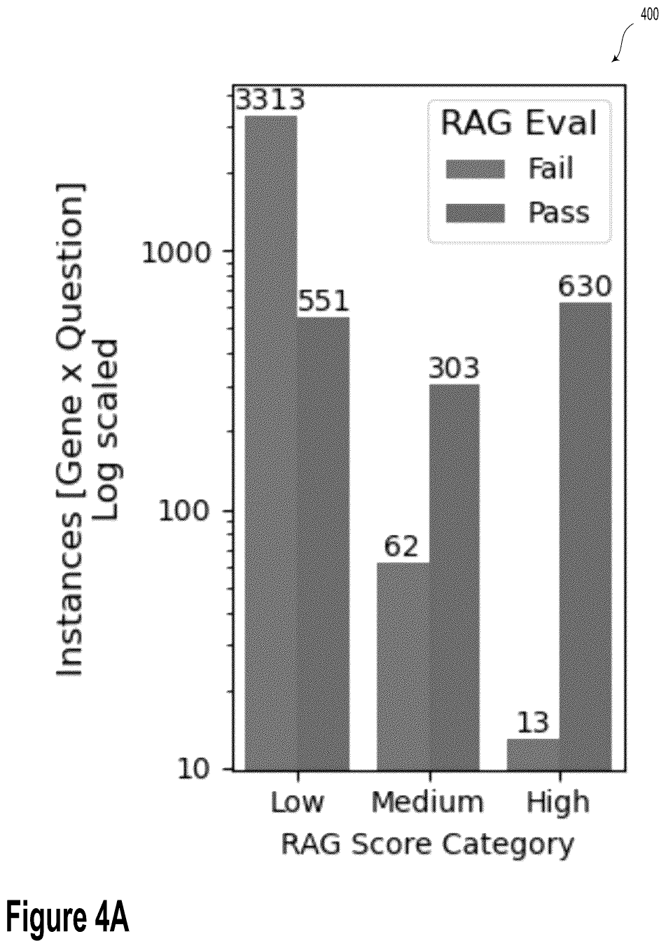

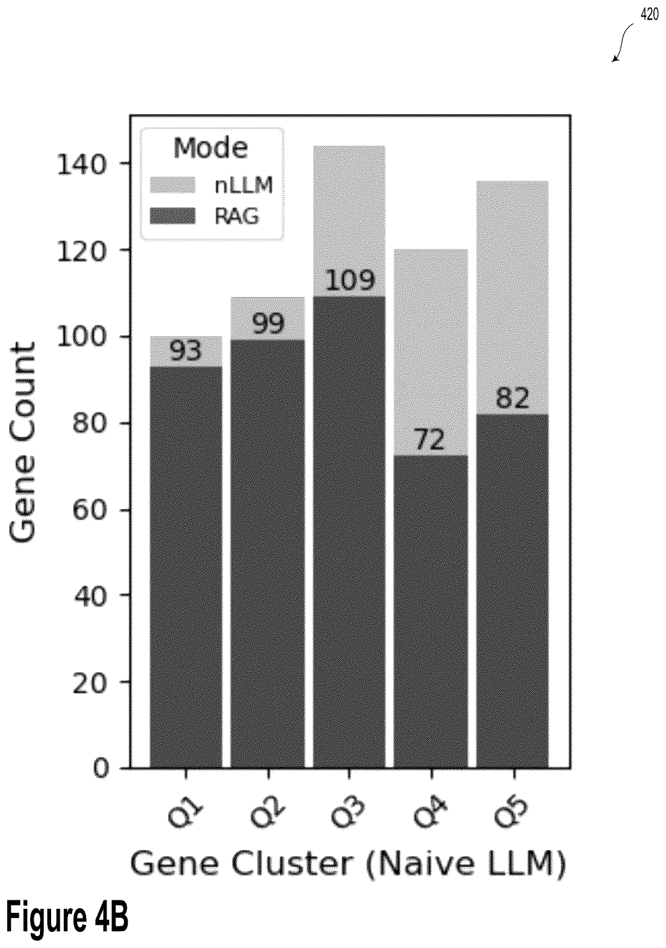

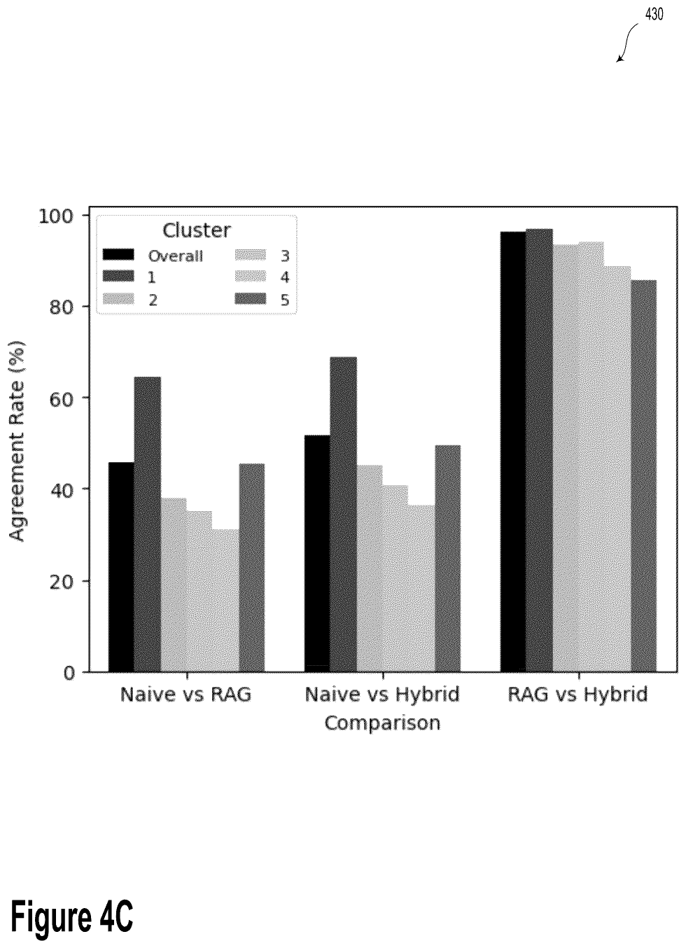

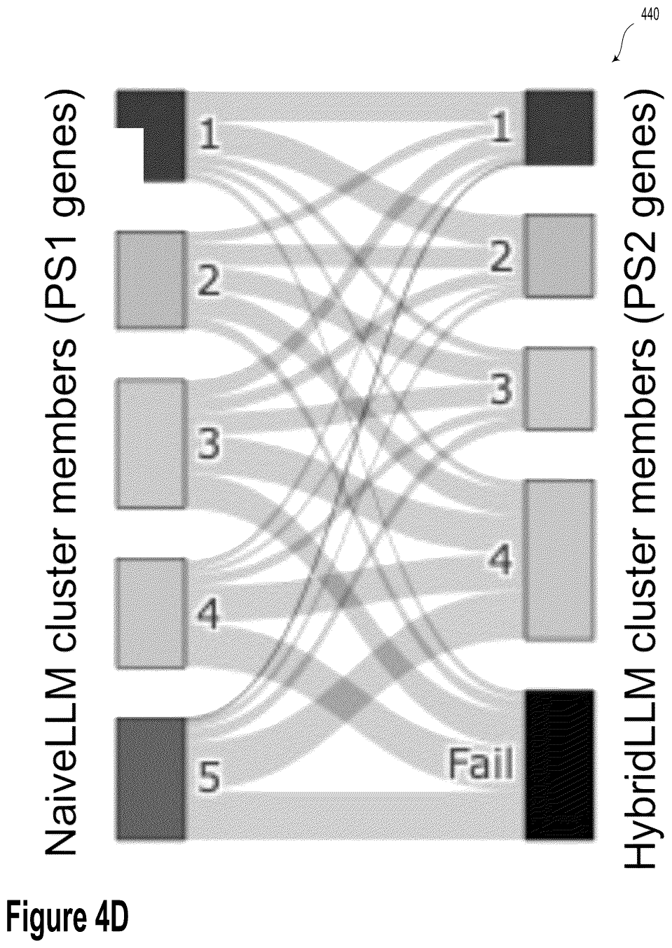

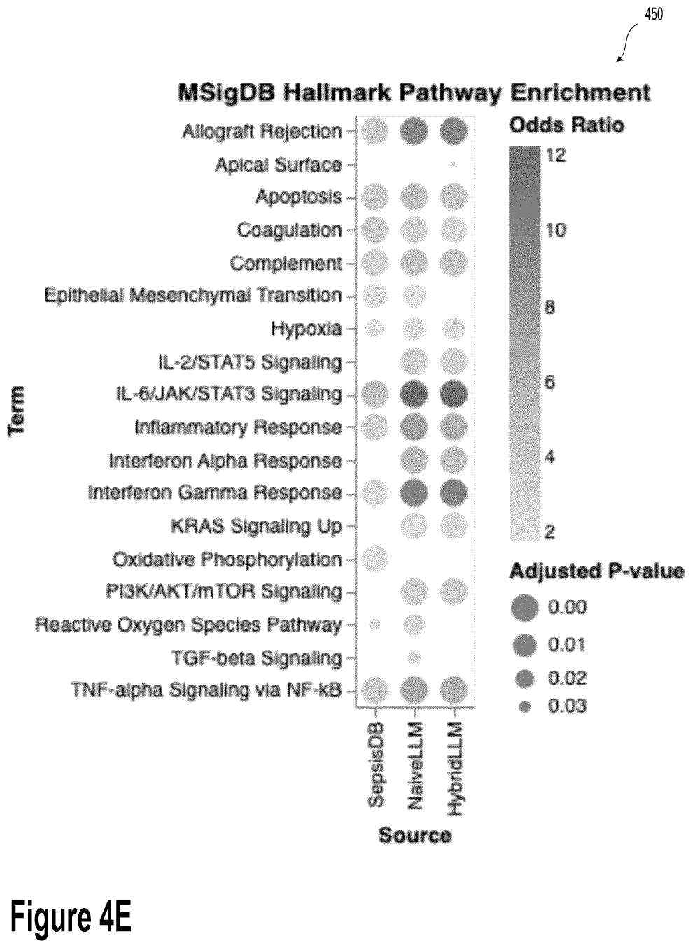

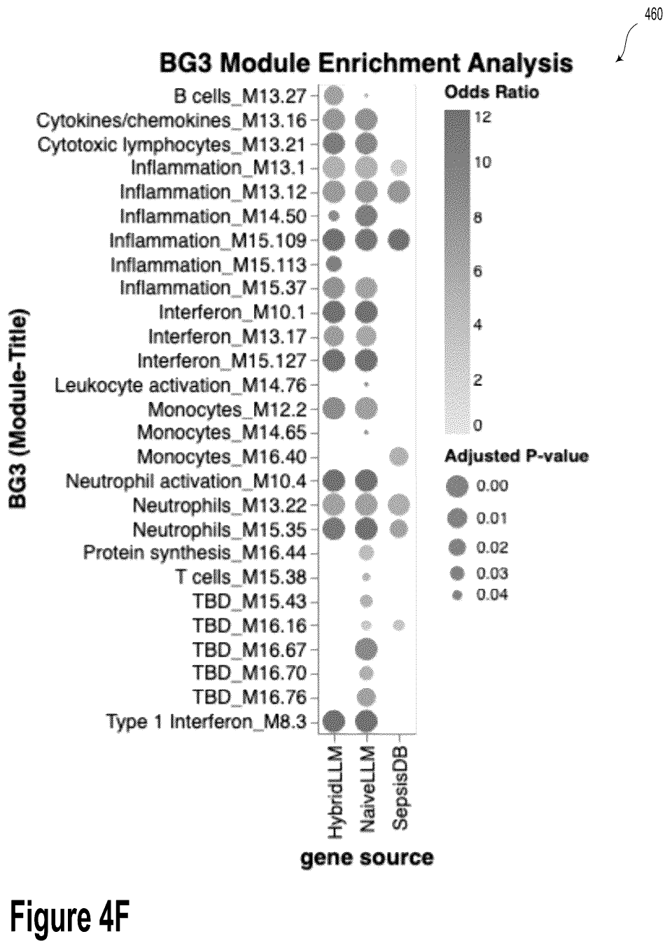

Of 4,872 queried instances, 1,484 passed the faithfulness test (30.5% pass rate), covering 455 unique genes designated as PS2 (FIG. 4A). The stringent pass rate reflects the conservative nature of literature-grounded evaluation, ensuring high-confidence literature support for retained genes. Analysis of our scoring rubric categories revealed that high-scoring instances demonstrated the strongest correlation with faithfulness pass rates, validating our scoring criteria and confirming that computational confidence metrics appropriately reflect literature evidence quality.

Cluster-Wise Recovery and Evidence Stratification. RAG evaluation revealed expected evidence-dependent recovery patterns across PS1 clusters (FIG. 4B). Top-tier clusters achieved highest literature validation rates: Q1 (93% recovery, 93/100 genes) and Q2 (90% recovery, 98/109 genes), consistent with their elevated baseline scores and extensive research documentation. Q3 showed moderate recovery (75%, 109/144 genes), while Q4 and Q5 both achieved approximately 60% recovery. This gradient confirms that higher computational scores correlate with stronger literature evidence availability, supporting the validity of our initial scoring framework (FIG. S7).

Method Agreement and Scoring Dynamics. Comparative analysis between evaluation approaches revealed moderate but systematic agreement patterns (FIG. 4C). Naive vs RAG comparison showed approximately 50% agreement, reflecting the conservative nature of literature-based filtering. Naive vs Hybrid demonstrated improved concordance, particularly for extreme-scoring genes where computational and literature evidence align. Cluster-wise agreement patterns showed expected conservation: Q1 exhibited maximum overlap due to consistently high-confidence literature support, while Q5 showed substantial agreement due to consistently limited evidence. Mid-tier clusters (Q2-Q4) displayed more variable agreement, indicating that literature context significantly influences evaluation outcomes for moderately evidenced genes (Table S6).

Re-clustering and Cluster Membership Dynamics. We re-clustered the 442 (filtered gene from RAG “pass” genes) using gene-wise weighted scores from HybridLLM, considering only the 1,484 RAG pass instances for scoring calculations. Following the same quantile-based clustering approach, we identified a maximum of 4 clusters for PS2, compared to 5 clusters in the original PS1 set, reflecting the more concentrated distribution of high-confidence, literature-validated genes.

The Sankey diagram reveals systematic shifts in cluster membership between NaiveLLM (PS1) and HybridLLM (PS2) approaches (FIG. 4D). The flow patterns demonstrate several key dynamics: Cluster consolidation is evident as genes from PS1's top clusters (Q1-Q2) predominantly flow into PS2's highest-tier clusters, indicating preserved high-confidence assignments. Upward mobility is observed where some genes from PS1's mid-tier clusters (Q2-Q3) migrate to higher PS2 clusters, suggesting that literature context enhanced their evidence profiles. Conservative filtering is apparent through the elimination of PS1's lowest-confidence cluster (Q5), with these genes either being filtered out entirely or redistributed into PS2's lower-tier clusters. The flow thickness patterns indicate that cluster retention is strongest for originally high-scoring genes, while cluster reassignment is most common for moderately scored genes where literature evidence significantly influenced final classification (Table S9).

Progressive Pathway Enrichment Through Filtering. Comparative functional enrichment analysis across the three gene sets reveals progressive enhancement of sepsis-relevant pathway signatures (FIG. 4E). While the evidence set (n=927) shows broad pathway coverage with moderate enrichment intensities, both PS1 (NaïveLLM, n=609) and PS2 (HybridLLM, n=442) demonstrate substantially higher odds ratios for core inflammatory pathways including complement activation, TNF-α signaling, inflammatory response, and interferon pathways. This progressive enrichment intensity suggests successful noise reduction and signal enhancement through computational filtering and literature validation.

BG3 module enrichment analysis further validates PS2's biological coherence by demonstrating specific enrichment for immune cell-type modules directly relevant to sepsis pathophysiology (FIG. 4F). PS2 genes show pronounced enrichment for inflammation-associated modules, interferon response pathways, monocyte activation signatures, and neutrophil activation modules. This cell-type specificity aligns with established sepsis immunopathology, where monocyte/macrophage dysfunction and neutrophil activation are central disease mechanisms. Importantly, PS2 demonstrates selective absence of enrichment for non-specific gene modules, indicating successful filtering of housekeeping and broadly expressed genes that lack sepsis-specific relevance.

PS2 maintains enrichment for most core sepsis pathways present in PS1, while showing selective loss of broader regulatory pathways including TGF-β signaling and epithelial mesenchymal transition (EMT). This filtering pattern, also observed in SepsisDB-derived gene sets, reflects the literature-validation process's preference for well-documented acute inflammatory processes over tissue remodeling and resolution mechanisms.

iv. Multi-Dimensional Optimization Identifies High Confidence Priority Set 3 and Candidates Genes

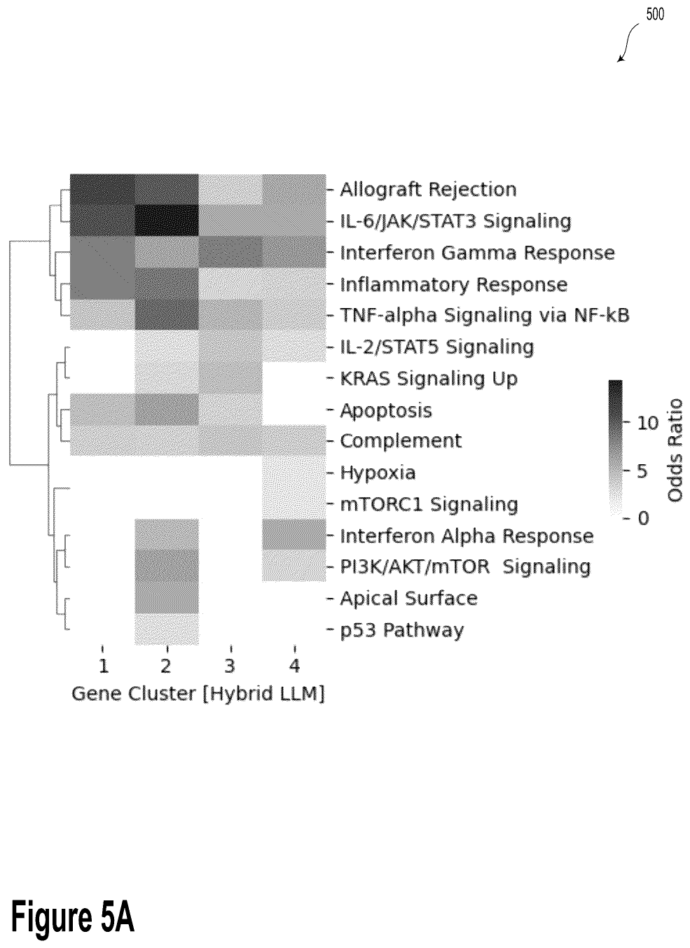

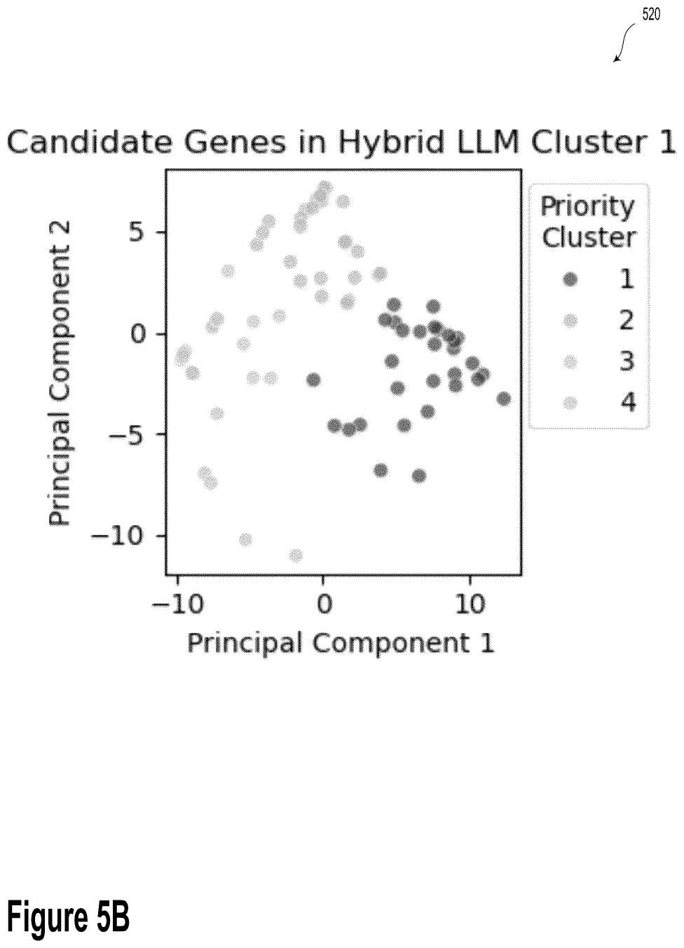

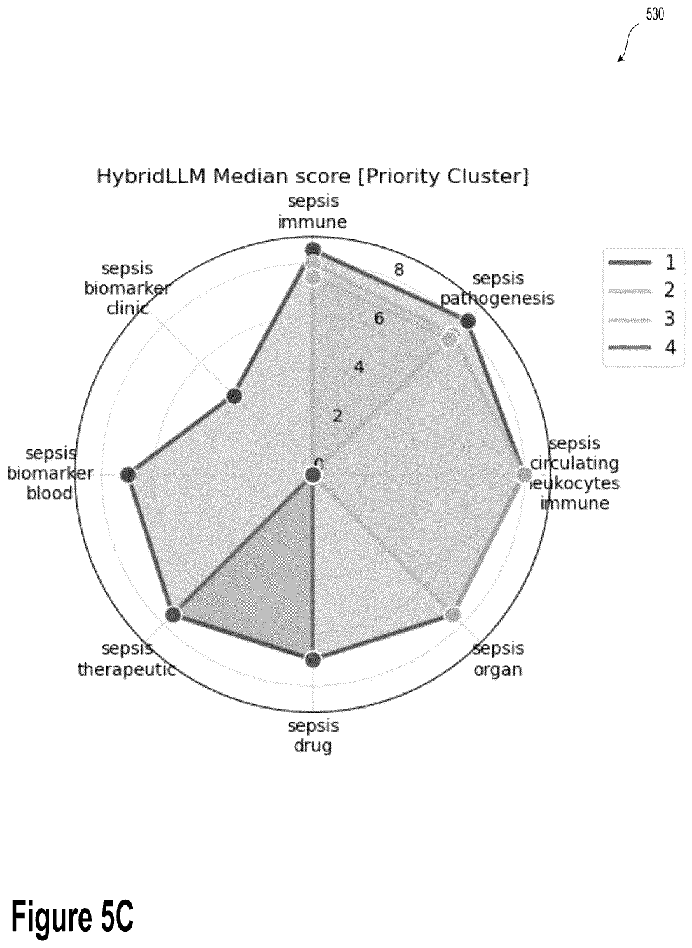

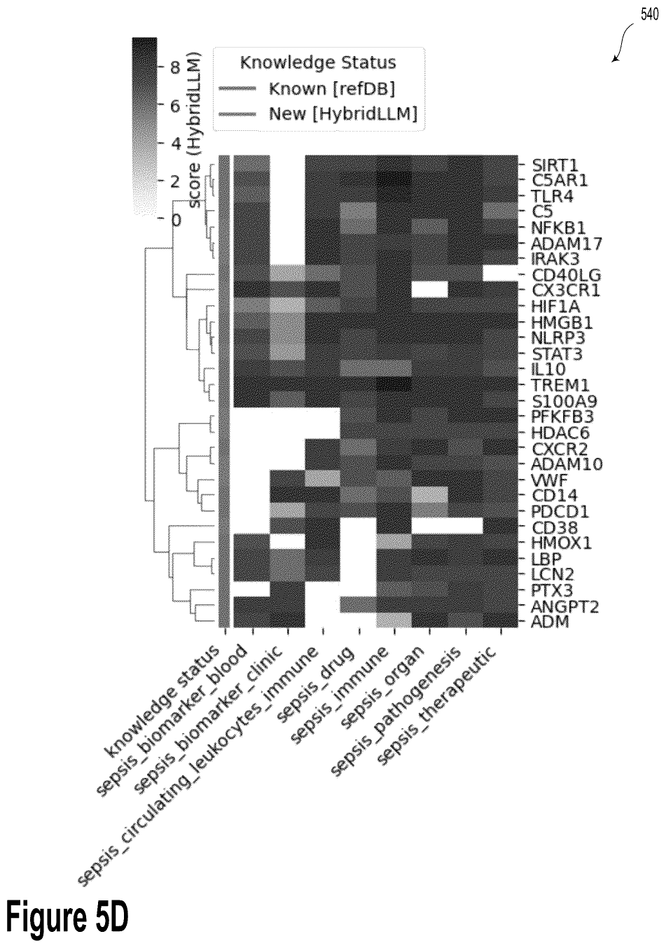

Cluster-Specific Functional Analysis and Final Prioritization. To identify the most promising therapeutic targets from PS2, we conducted cluster-specific functional enrichment analysis followed by multi-dimensional scoring optimization. Initial analysis of PS2's four clusters using MSigDB revealed distinct functional specialization patterns (FIG. 5A). The top performing cluster (Cluster 1) demonstrated the strongest enrichment across core sepsis pathways including allograft rejection, IL-6/JAK/STAT3 signaling, interferon gamma response, inflammatory response, and TNF-α signaling via NF-κB, establishing it as the highest-confidence functional cluster. We designated this gene set as Priority set 3 (PS3 n=82 genes: Table S9, S10).

Multi-Dimensional Score Analysis and Sub-Clustering. We selected PS3 for detailed multi-dimensional analysis using principal component analysis (PCA) across HybridLLM scores for all eight evaluation categories (FIG. 5B). PCA revealed four distinct sub-clusters within this high-confidence gene set, indicating further functional or evidence-quality stratification. The PCA clustering captured genes with similar scoring profiles across the multi-dimensional evaluation space, allowing identification of the most consistently high-performing candidates. Comparative analysis using polar plots revealed distinct scoring profiles across the four PCA-derived clusters (FIG. 5C). The radar chart comparison of mean Hybrid scores across eight evaluation dimensions demonstrated that PCA Cluster 1 genes consistently achieved the highest scores across all evaluation criteria, including pathogenesis association, immune response relevance, clinical biomarker potential, and therapeutic targeting feasibility. This comprehensive high performance across multiple independent evaluation dimensions established PCA Cluster 1 as the optimal candidate set (Table S10). This analysis established a clear hierarchy: PS2 (455 genes)→PS3 (82 genes from top cluster)→final candidate set (30 genes from optimal PCA cluster).

Based on the multi-dimensional optimization analysis of PS3 genes (n=82), we designated the 30 genes from the top-performing PCA as candidate set, representing high confidence sepsis genes. The comprehensive scoring heatmap reveals candidate set's composition and validation status (FIG. 5D, Table S10): genes are color-coded to distinguish between established candidates from our original evidence set (n=19 “Known”) and novel discoveries identified through our computational pipeline (n=11 “New”). All candidate genes demonstrate consistently high HybridLLM scores across the eight evaluation queries, with both known and newly identified genes showing comparable scoring profiles. Importantly, each candidate gene is backed by literature-grounded evidence from our RAG system and underwent additional verification through deep-search features to ensure robust evidence support (Supplementary Method 2).

Discovery and Validation Balance. The final candidate set successfully balances validation and discovery objectives by including both established sepsis genes that serve as positive controls and novel candidates that represent potential breakthrough targets. The comparable scoring patterns between known and new genes validates our computational framework's ability to identify clinically relevant candidates while the literature grounding and deep-search verification ensures that novel discoveries possess substantial evidence support rather than representing computational artifacts.

This progressive filtering approach—from 10,824 genome-wide genes to 30 ultra-high confidence candidates-demonstrates successful integration of computational assessment, literature validation, and multi-dimensional optimization to identify the most promising sepsis therapeutic targets for immediate experimental investigation.

### D. Discussion

The benchmarking validation demonstrates systematic biological coherence through 71.2% recall with strong evidence quality correlation, distinguishing our approach from routine LLM applications that lack rigorous validation against expert-curated standards. RAG's conservative behavior-frequent zero assignments with selective enhancement only when literature support exists-reflects appropriate uncertainty handling for biomedical inference where false discoveries incur significant costs. The systematic blind spots in metabolic pathways (protein synthesis, urea cycle, cellular maintenance) reveal coherent framework limitations that likely reflect literature emphasis on inflammatory over metabolic mechanisms in sepsis research, enabling informed large-scale result interpretation.

Recent biomedical LLM applications reveal fundamental limitations requiring sophisticated solutions. Kim et al. (2024) achieved only 16.0% accuracy with GPT-4 for phenotype-driven gene prioritization, significantly underperforming traditional bioinformatics tools. Recent RAG implementations demonstrate incremental improvements but lack comprehensive validation. GeneRAG (2024) achieved 39% improvement in gene question answering through Maximal Marginal Relevance integration, while MIRAGE benchmarking showed medical RAG systems improving LLM accuracy by up to 18%. However, these approaches focus on general question-answering rather than systematic gene prioritization workflows. Wu et al. (2025) developed RAG-driven Chain-of-Thought methods for rare disease diagnosis from clinical notes, achieving over 40% top-10 accuracy, but without systematic faithfulness evaluation or biological validation. AlzheimerRAG demonstrated multimodal integration for literature retrieval, and DRAGON-AI applied RAG to ontology generation, but none address the core challenges of evidence verification and systematic validation in clinical contexts.

Early literature-based gene prioritization was established by Génie (2011), which ranked genes using MEDLINE abstracts and ortholog information but relied on basic text mining without modern NLP techniques. Established methods like ProGENI (protein-protein interaction integration) and the Monarch Initiative (curated database integration) demonstrate superior performance through structured knowledge but lack dynamic literature access and evidence verification capabilities that modern biomedical research demands.

Our framework addresses these limitations through systematic integration of curated domain knowledge (6,346 high-quality sepsis publications), dual-evaluator faithfulness assessment (71.94% inter-evaluator agreement), and Chain-of-Thought hybrid reasoning. The resulting 71.2% benchmark recall substantially exceeds recent LLM applications while maintaining biological coherence through systematic pathway enrichment validation.

The three-stage filtering approach successfully reduced computational noise while preserving biological signal, as evidenced by progressive pathway enrichment from PS1 (n=609) to PS2 (n=442) to PS3 (n=82). The 94% filtering efficiency demonstrates appropriate stringency, though the 30.5% RAG faithfulness pass rate may introduce bias toward well-studied genes. The final candidate gene composition (19 known, 11 novel candidates) with comparable scoring profiles supports balanced discovery-validation dynamics while avoiding computational artifacts.

Strong enrichment for sepsis-relevant pathways (TNF-α signaling, complement activation, interferon responses) confirms successful biological signal capture. The quantile-based functional stratification validates score-based prioritization, with higher-scoring clusters demonstrating stronger inflammatory pathway enrichment. However, selective loss of broader regulatory pathways (TGF-0 signaling, epithelial mesenchymal transition) in literature-validated sets suggests potential under-representation of resolution mechanisms.

The 74% agreement between GPT-4o and GPT-4T versions provides moderate confidence in framework robustness across model iterations. This consistency level highlights the sensitivity of LLM-based approaches to model architecture differences. The systematic correlation between computational scores and PubMed publication frequency validates evidence-weighted behavior but reveals potential circularity favoring well-published genes.

The framework's computational requirements present both advantages and limitations. Multi-stage evaluation requires substantial API costs and processing time that may limit accessibility. The dependency on proprietary models creates sustainability concerns for long-term research applications and reproducibility as models evolve.

Future iterations should incorporate experimental data beyond literature analysis, including functional genomics and clinical outcomes data. Integration of tissue-specific and temporal expression patterns could enhance context-relevant prioritization. Expanding beyond inflammatory mechanisms to include metabolic and repair pathways would provide more comprehensive target coverage.

The methodological framework provides a foundation for systematic AI-driven biomedical discovery with potential applications beyond sepsis research. However, continued validation against experimental outcomes and clinical data will be essential for establishing real-world utility and addressing the persistent challenge of translating computational predictions into therapeutic advances. The framework's strength lies in systematic prioritization rather than definitive target identification, offering improved resource allocation for early-stage research while acknowledging the substantial experimental validation required for clinical translation.

### E. CONCLUSIONS

We developed and validated a comprehensive computational framework for systematic gene prioritization in sepsis research, addressing critical limitations in current LLM-based biomedical applications. Through progressive filtering of 10,824 genes to 30 ultra-high confidence candidates, our approach demonstrates that rigorous methodology can transform unreliable LLM outputs into systematically validated biological insights. The framework's key innovations include curated domain-specific knowledge base construction, dual-evaluator faithfulness assessment to reduce hallucination risks, and Chain-of-Thought hybrid reasoning that synthesizes computational predictions with literature evidence. Benchmark validation against expert-curated databases achieved 71.2% recall with strong evidence quality correlation, substantially exceeding recent LLM applications while maintaining biological coherence through systematic pathway enrichment. The identification of 11 novel high-confidence candidates alongside 19 established sepsis genes demonstrate balanced discovery-validation dynamics, though these candidates represent systematically prioritized hypotheses requiring extensive experimental validation rather than immediate therapeutic targets.

Our methodological approach combining computational assessment, literature validation, and multi-dimensional optimization-provides a template for evaluating AI-driven biomedical discovery tools while establishing principles for responsible AI application in biomedical research. The modular design allows flexible adaptation where LLM models, gene lists, evaluation queries, and RAG databases can be systematically replaced to address any user-defined disease context or research question, extending applicability beyond sepsis to diverse biomedical domains. The systematic characterization of framework limitations, particularly blind spots in metabolic pathways and bias toward well-studied genes, enables informed interpretation of results and guides future improvements. While computational efficiency and model dependency present adoption challenges, this work demonstrates that sophisticated computational frameworks can bridge the gap between AI capabilities and clinically translatable applications, provided they incorporate rigorous validation, systematic bias characterization, and appropriate uncertainty handling. The framework's strength lies in efficient resource allocation for early-stage research, offering a pathway for harnessing LLM potential in biomedical research while maintaining the scientific rigor essential for advancing from computational predictions to therapeutic breakthroughs.

### V. EXAMPLE METHODS

The following description and accompanying drawings will elucidate features of various example embodiments. The embodiments provided are by way of example and are not intended to be limiting. As such, the dimensions of the drawings are not necessarily to scale.

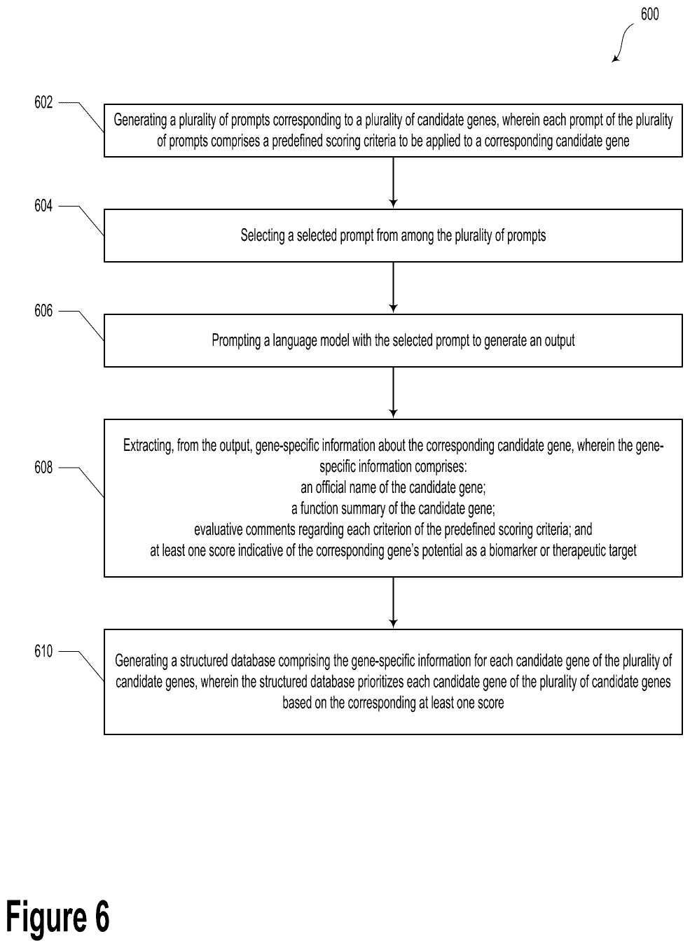

FIG. 6 illustrates a method 600 for automated candidate gene prioritization, according to example embodiments. It will be understood that the method 600 can include fewer or more steps or blocks than those expressly illustrated or otherwise disclosed herein. Furthermore, respective steps or blocks of method 600 can be performed in any order and each step or block can be performed one or more times. In some embodiments, some or all of the blocks or steps of method 600 can be carried out by controller 150 and/or other elements of computational framework 100 as illustrated and described in relation to FIG. 1.

Block 602 includes generating a plurality of prompts corresponding to a plurality of candidate genes. Each prompt of the plurality of prompts comprises a predefined scoring criteria to be applied to a corresponding candidate gene.

In various examples, the predefined scoring criteria could be based on developing a targeted assay for monitoring patients with sepsis. In such scenarios, the predefined scoring criteria may correspond to: the candidate gene's relevance to sepsis pathogenesis, host immune response to the candidate gene, the candidate gene's association with organ dysfunction, the candidate gene's biomarker potential, and/or the candidate gene's therapeutic implications for managing sepsis.

In some embodiments, the plurality of candidate genes may be selected from module M10.1 of the BloodGen3 repertoire.

Block 604 includes selecting a selected prompt from among the plurality of prompts.

Block 606 includes prompting a language model with the selected prompt to generate an output.

Block 608 includes extracting, from the output, gene-specific information about the corresponding candidate gene. In an example embodiment, the gene-specific information includes: an official name of the candidate gene, a function summary of the candidate gene, evaluative comments regarding each criterion of the predefined scoring criteria, and at least one score indicative of the corresponding gene's potential as a biomarker or therapeutic target.

In some examples, the gene-specific information may include at least one of: the corresponding gene's association with different types of interferon responses, relevance to circulating leukocytes immune biology, current use as a biomarker in clinical settings, potential value as a blood transcriptional biomarker, known drug target status; and therapeutic relevance for diseases involving the immune system.

Block 610 includes generating a structured database comprising the gene-specific information for each candidate gene of the plurality of candidate genes, wherein the structured database prioritizes each candidate gene of the plurality of candidate genes based on the corresponding at least one score.

In some examples, method 600 includes displaying, via a graphical user interface, a set of high-priority candidate genes and their corresponding at least one scores. Additionally or alternatively, method 600 may include initiating a candidate validation study of the high-priority candidate genes.

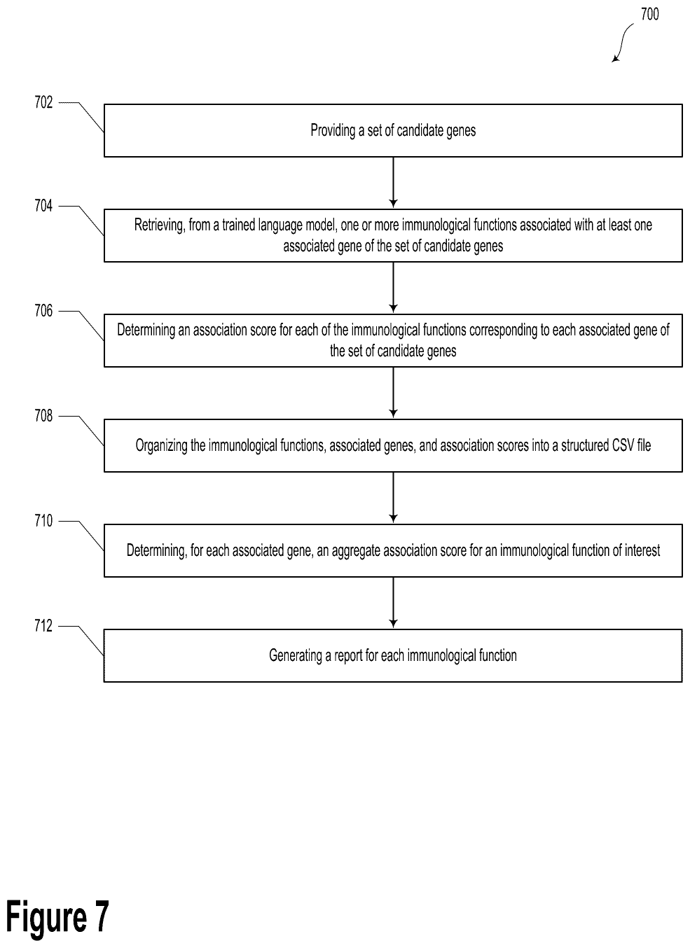

FIG. 7 illustrates a method 700, according to example embodiments. It will be understood that the method 700 can include fewer or more steps or blocks than those expressly illustrated or otherwise disclosed herein. Furthermore, respective steps or blocks of method 700 can be performed in any order and each step or block can be performed one or more times. In some embodiments, some or all of the blocks or steps of method 700 can be carried out by controller 150 and/or other elements of computational framework 100 as illustrated and described in relation to FIG. 1.

Block 702 of method 700 includes providing a set of candidate genes.

Block 704 includes retrieving, from a trained language model, one or more immunological functions associated with at least one associated gene of the set of candidate genes.

In some examples, retrieving the one or more immunological functions includes prompting the trained model at least three times to capture all immunological functions for each gene of the set of candidate genes. In such scenarios, the method 700 also includes consolidating respective outputs from the trained model in response to the at least three prompts.

Block 706 includes determining an association score for each of the immunological functions corresponding to each associated gene of the set of candidate genes.

Block 708 includes organizing the immunological functions, associated genes, and association scores into a structured CSV file;

Block 710 includes determining, for each associated gene, an aggregate association score for an immunological function of interest. In some examples, determining the aggregate association score could be based on determining a strength of an association between a given gene of the set of candidate genes and the given immunological function. Yet further, in some examples, determining the strength of the association between the given gene and the given immunological function is based on a survey of scientific studies and meta-analyses. In some examples, determining the aggregate association score comprises summing the association scores for the immunological function of interest.

Block 712 includes generating a report for each immunological function. In some examples, generating the report includes generating a summary table. In some embodiments, the summary table includes gene symbols, immunological functions, association scores, and narratives. In such scenarios, the narratives are based on peer-reviewed scientific knowledge and include specific roles the given genes are known to play in an immune system. In some examples, generating the report includes identifying cell types associated with the candidate genes and corresponding immunological functions. Additionally or alternatively, generating the report includes identifying transcriptional programs associated with the candidate genes and corresponding immunological functions. In other examples, generating the report could include generating a narrative describing functional convergences among the candidate genes and corresponding immunological functions.

In various examples, method 700 could also include confirming, using a trained language model, one or more outputs of the method, providing one or more source references corresponding to the one or more outputs of the method, and/or generating, using a trained language model, one or more summary tables.

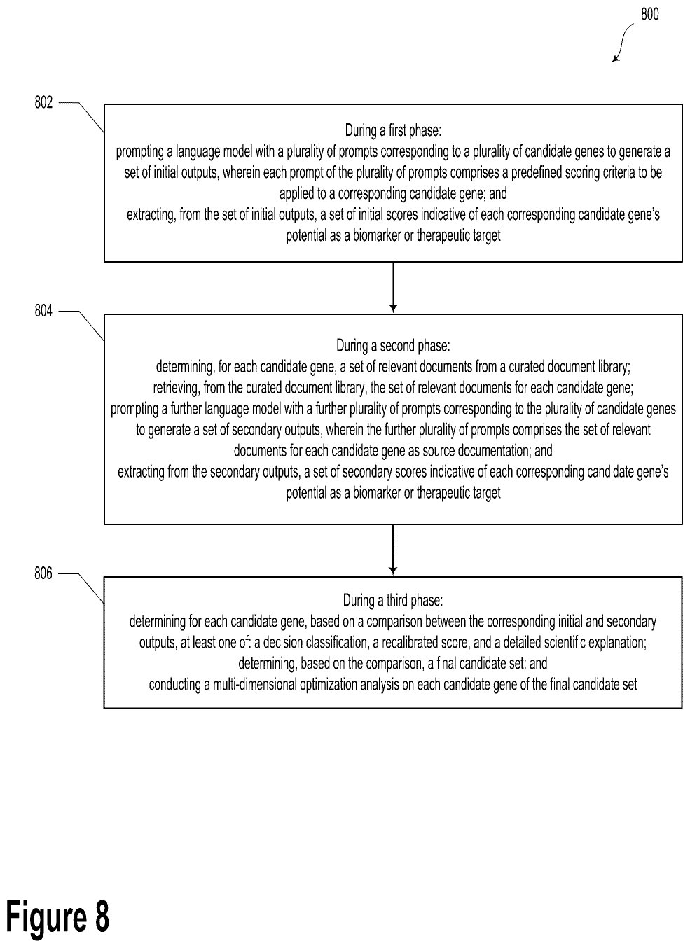

FIG. 8 illustrates a method 800, according to example embodiments. It will be understood that the method 800 can include fewer or more steps or blocks than those expressly illustrated or otherwise disclosed herein. Furthermore, respective steps or blocks of method 800 can be performed in any order and each step or block can be performed one or more times. In some embodiments, some or all of the blocks or steps of method 800 can be carried out by controller 150 and/or other elements of computational framework 100 as illustrated and described in relation to FIG. 1.

Block 802 includes, during a first phase, prompting a language model with a plurality of prompts corresponding to a plurality of candidate genes to generate a set of initial outputs. Each prompt of the plurality of prompts comprises a predefined scoring criteria to be applied to a corresponding candidate gene. Block 802 also includes extracting, from the set of initial outputs, a set of initial scores indicative of each corresponding candidate gene's potential as a biomarker or therapeutic target.

In some examples, the predefined scoring criteria include at least one of: the candidate gene's relevance to pathogenesis of a target pathology, host immune response to the candidate gene, the candidate gene's biomarker potential, and the candidate gene's drug target feasibility.

Block 804 includes, during a second phase, determining, for each candidate gene, a set of relevant documents from a curated document library and retrieving, from the curated document library, the set of relevant documents for each candidate gene. Block 804 also includes prompting a further language model with a further plurality of prompts corresponding to the plurality of candidate genes to generate a set of secondary outputs. The further plurality of prompts includes the set of relevant documents for each candidate gene as source documentation. Block 804 yet further includes extracting from the secondary outputs, a set of secondary scores indicative of each corresponding candidate gene's potential as a biomarker or therapeutic target.

Block 806 includes, during a third phase, determining for each candidate gene, based on a comparison between the corresponding initial and secondary outputs, at least one of: a decision classification, a recalibrated score, and a detailed scientific explanation. Block 806 further includes determining, based on the comparison, a final candidate set and conducting a multi-dimensional optimization analysis on each candidate gene of the final candidate set.

In some example embodiments, the predefined scoring criteria may include pathogenesis association, host immune response, organ dysfunction, circulating leukocyte biology, clinical biomarker use, blood transcriptional biomarker potential, drug target status, and therapeutic relevance.

In various examples, conducting the multi-dimensional optimization analysis may include applying principal component analysis (PCA) to each of the predefined scoring criteria.

### VI. CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, operation, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step, block, or operation that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer-readable medium such as a storage device including random-access memory (RAM), a disk drive, a solid-state drive, or another storage medium.

The computer-readable medium can also include non-transitory computer-readable media such as computer-readable media that store data for short periods of time like register memory and processor cache. The computer-readable media can further include non-transitory computer-readable media that store program code and/or data for longer periods of time. Thus, the computer-readable media may include secondary or persistent long-term storage, like read-only memory (ROM), optical or magnetic disks, solid state drives, compact-disc read-only memory (CD-ROM), for example. The computer-readable media can also be any other volatile or non-volatile storage systems. A computer-readable medium can be considered a computer-readable storage medium, for example, or a tangible storage device.

Moreover, a step, block, or operation that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed:

1. A method for automated candidate gene prioritization, the method comprising:
generating a plurality of prompts corresponding to a plurality of candidate genes, wherein each prompt of the plurality of prompts comprises a predefined scoring criteria to be applied to a corresponding candidate gene;
selecting a selected prompt from among the plurality of prompts;
prompting a language model with the selected prompt to generate an output;
extracting, from the output, gene-specific information about the corresponding candidate gene, wherein the gene-specific information comprises:
an official name of the candidate gene;
a function summary of the candidate gene;
evaluative comments regarding each criterion of the predefined scoring criteria; and
at least one score indicative of the corresponding gene's potential as a biomarker or therapeutic target; and
generating a structured database comprising the gene-specific information for each candidate gene of the plurality of candidate genes, wherein the structured database prioritizes each candidate gene of the plurality of candidate genes based on the corresponding at least one score.

2. The method of claim 1, further comprising at least one of:
displaying, via a graphical user interface, a set of high-priority candidate genes and their corresponding at least one scores; or
initiating a candidate validation study of the high-priority candidate genes.

3. The method of claim 1, wherein the predefined scoring criteria are based on developing a targeted assay for monitoring patients with sepsis, and wherein the predefined scoring criteria correspond to:
the candidate gene's relevance to sepsis pathogenesis;
host immune response to the candidate gene;
the candidate gene's association with organ dysfunction;
the candidate gene's biomarker potential; and
the candidate gene's therapeutic implications for managing sepsis.

4. The method of claim 3, wherein the plurality of candidate genes is selected from module M10.1 of the BloodGen3 repertoire.

5. The method of claim 3, wherein the plurality of candidate genes is selected from the BloodGen3 repertoire.

6. The method of claim 1, wherein the gene-specific information further comprises at least one of:
the corresponding gene's association with different types of interferon responses;
relevance to circulating leukocytes immune biology;
current use as a biomarker in clinical settings;
potential value as a blood transcriptional biomarker; or
known drug target status; and therapeutic relevance for diseases involving the immune system.

7. A method comprising, providing a set of candidate genes;
retrieving, from a trained language model, one or more immunological functions associated with at least one associated gene of the set of candidate genes;
determining an association score for each of the immunological functions corresponding to each associated gene of the set of candidate genes;
organizing the immunological functions, associated genes, and association scores into a structured CSV file;
determining, for each associated gene, an aggregate association score for an immunological function of interest; and
generating a report for each immunological function.

8. The method of claim 7, wherein retrieving the one or more immunological functions comprises prompting the trained language model at least three times to capture all immunological functions for each gene of the set of candidate genes, and wherein the method further comprises consolidating respective outputs from the trained language model in response to the at least three prompts.

9. The method of claim 7, wherein determining the aggregate association score is based on determining a strength of an association between a given gene of the set of candidate genes and the given immunological function.

10. The method of claim 9, wherein determining the strength of the association between the given gene and the given immunological function is based on a survey of scientific studies and meta-analyses.

11. The method of claim 7, wherein determining the aggregate association score comprises summing the association scores for the immunological function of interest.

12. The method of claim 7, wherein generating the report comprises generating a summary table, wherein the summary table comprises gene symbols, immunological functions, association scores, and narratives, wherein the narratives are based on peer-reviewed scientific knowledge and include specific roles the given genes are known to play in an immune system.

13. The method of claim 7, wherein generating the report comprises identifying cell types associated with the candidate genes and corresponding immunological functions.

14. The method of claim 7, wherein generating the report comprises identifying transcriptional programs associated with the candidate genes and corresponding immunological functions.

15. The method of claim 7, wherein generating the report comprises generating a narrative describing functional convergences among the candidate genes and corresponding immunological functions.

16. The method of claim 7, further comprising:
confirming, using a trained language model, one or more outputs of the method;
providing one or more source references corresponding to the one or more outputs of the method; and
generating, using a trained language model, one or more summary tables.

17. A method comprising:
during a first phase:
prompting a language model with a plurality of prompts corresponding to a plurality of candidate genes to generate a set of initial outputs, wherein each prompt of the plurality of prompts comprises a predefined scoring criteria to be applied to a corresponding candidate gene; and
extracting, from the set of initial outputs, a set of initial scores indicative of each corresponding candidate gene's potential as a biomarker or therapeutic target;
during a second phase:
determining, for each candidate gene, a set of relevant documents from a curated document library;
retrieving, from the curated document library, the set of relevant documents for each candidate gene;
prompting a further language model with a further plurality of prompts corresponding to the plurality of candidate genes to generate a set of secondary outputs, wherein the further plurality of prompts comprises the set of relevant documents for each candidate gene as source documentation; and
extracting from the secondary outputs, a set of secondary scores indicative of each corresponding candidate gene's potential as a biomarker or therapeutic target; and
during a third phase:
determining for each candidate gene, based on a comparison between the corresponding initial and secondary outputs, at least one of: a decision classification, a recalibrated score, and a detailed scientific explanation;
determining, based on the comparison, a final candidate set; and
conducting a multi-dimensional optimization analysis on each candidate gene of the final candidate set.

18. The method of claim 17, wherein the predefined scoring criteria comprise at least one of:
the candidate gene's relevance to pathogenesis of a target pathology;
host immune response to the candidate gene;
the candidate gene's biomarker potential; and
the candidate gene's drug target feasibility.

19. The method of claim 18, wherein the predefined scoring criteria comprise:
pathogenesis association;
host immune response;
organ dysfunction;
circulating leukocyte biology;
clinical biomarker use;
blood transcriptional biomarker potential;
drug target status; and
therapeutic relevance.

20. The method of claim 19, wherein conducting the multi-dimensional optimization analysis comprises applying principal component analysis (PCA) to each of the predefined scoring criteria.
